(19) United States
(12) Patent Application Publication
(10) Pub. No.: US 2011/0052730 A1
(43) Pub. Date: Mar. 3, 2011

(54) Leaves Extract of Panax sp., a Process of Making the Same and Uses Thereof

(75) Inventors: Sung Sick Woo, Seoul (KR); Dong Seon Kim, Daejeon (KR); Seon Gil Do, Seoul (KR); Young Chul Lee, Daejeon (KR); Jong Han Kim, Chungcheongbuk-do (KR); Soo Kyung Sung, Chungcheongbuk-do (KR)

(73) Assignee: UNIGEN, INC., Chungcheongnam-do (KR)

(21) Appl. No.: 12/866,859

(22) PCT Filed: Feb. 18, 2009
(86) PCT No.: PCT/KR09/00778

§ 371 Date: Nov. 12, 2010

(30) Foreign Application Priority Data
Feb. 19, 2008 (KR) 10-2008-0014881
Feb. 19, 2008 (KR) 10-2008-0014886
Feb. 19, 2008 (KR) 10-2008-0014887

(51) Int. Cl. A61K 36/254; A61K 31/704; A61P 21/00

(52) U.S. Cl. 424/728; 514/26

(57) ABSTRACT

The present invention relates to a composition for improvement of exercise performance, fatigue recovery or prevention of oxidation response comprising Panax species plant leaves extract or processed product of the leaves extract, or mixture of the both as an active ingredient. The present composition comprising Panax species plant leaves extract or processed product of the leaves extract, or mixture of the both increases the exercise performance, inhibit the accumulation of fatigue markers in blood and prevents oxidation response, and thus is useful to improve physical strength and exercise capacity.

### TECHNICAL FIELD

This research was supported by a grant (code #PF0321204-00) from Plant Diversity Research Center of 21st Century Frontier Research Program funded by Ministry of Science and Technology of Korean government.

The present invention relates to a composition for improvement of exercise performance, fatigue recovery, and prevention of oxidation response comprising Panax species plant leaves extract or processed Panax species plant leaves extract, or mixture of the both as an active ingredient.

### BACKGROUND ART

Generally, if muscles do not move continuously, the function of muscle becomes lowered with aging, and the muscular volume and neuromuscular junction (motor unit) decrease, resulting in fatigue, enervation and vitality reduction, and in the end, the quality of life becomes significantly worse (Dohergy T J, J Appl. Physiol., 95:1717-1727, 2003; Eric E, et al., Physiol. Behav., 92(1-2): 129-135, 2007).

To prevent such problems, it is recommended that appropriate exercises such as resistance training be performed continuously, along with a proper dietary treatment. However, busy people today rather desire to receive a help of dietary supplement including ginseng and red ginseng which has been known as having an effect of nourishing vigorousness.

Regular exercise has become a part of life in order for modern people to improve their quality of life. Not only sportsmen but ordinary people want more energy and endurance in their daily lives. Ginseng root extract in various formulations of a dietary supplement is one of the candidates for which many scientific studies have been conducted to prove the efficacy of ginseng in elevation of physical performance.

Panax ginseng has been regarded as a natural ergogenic aid for a long time and it is also known to be good for vigorousness, anti-oxidation and hangover (Kim S H, et al., J Sports Med. Phys. Fitness., 45(2):178-82, 2005). In particular, Panax ginseng has been known to improve mitochondrial energy metabolism, and ginsenosides Rg1 and Rb1 are known to enhance the aerobic exercise performance (Wang L C and Lee T F, Planta Med., 64(2):130-133, 1998). It also has been reported that anti-oxidation effect of ginsenosides Rg3 and Re, which have been known as active ingredients of ginseng, reduces oxidative stress (Tian J, et al., Neurosci. Lett., 374(2):92-97, 2005; Cho W C, et al., Eur. J. Pharmacol., 550(1-3):173-179, 2006). And, ginseng has been reported to decrease skeletal muscle cell membrane damage by reducing the leakage of plasma creatine kinase (CK) during a very intensive exercise (Hsu C C, et al., World J. Gastroenterol., 11(34):5327-5331, 2005). The pharmacological actions of ginseng are presumed to be involved in anti-aging, immune enhancement, anti-tumor, anti-stress, anti-oxidation and organ protective effects (Gillis C N, Biochem Pharmacol., 54(1):1-8, 1997; Attele A S, et al., Biochem Pharmacol., 58(11):1685-93, 1999; Shin H R, et al., Cancer Causes Control., 11(6):565-76, 2000).

Ginseng root has been used as an ergogenic aid for endurance exercise. It has been ingested by many athletes in the world in order to improve stamina and to facilitate rapid recovery from injuries. Ginseng root increases exercise duration time until exhaustion, decreases Malondialdehyde (MDA) and catalase (CAT) and increases superoxide dismutase (SOD). It was reported that the activities of CAT and SOD as scavenger enzymes were increased after ginseng root ingestion (2 g each times, 3 times day in sedentary humans) while MDA level was decreased (J. Sports Med Phys Fitness. 2005, 45(2): 178-82).

Panax notoginseng root also improves exercise endurance time until exhaustion (J Strength Cond Res., 2005 19(1): 108-14). Ginseng root has been reported to improve pulmonary functions and exercise capacity in patients with Chronic Obstructive Pulmonary Disease (COPD) (Monaldi Arch Chest Dis. 2002, 57 (5-6): 242-6). Red ginseng root increases treadmill running time until exhaustion and inhibits exercise-induced increase in serotonin synthesis and tryptophan hydroxylase expression. It means that red ginseng shows a suppressive effect on serotonin level during exercise and thus ingestion of red ginseng root can function as an ergogenic mechanism (J. Pharmacol Sci. 2003, 93(2): 218-21).

Panax ginseng leaves have been reported as having anti-oxidant, hypoglycemic properties. It can suppress a sudden increase of glucose levels in blood and consequently it can decrease TBARS level in diabetic rats (J Ethnopharmacol. 2005 98 (3): 245-50). American ginseng leaves also have been reported to have an anti-hyperglycemic and thermogenic activities (Pharmacol Res., 2004, 49(2): 113-7).

However, there have been few cases in which clinical evidences support that physical endurance performance is improved by ingestion of dietary ginseng products (J Am Coll Nutr 1998, 17: 462-6, Int J Sport Nutr 1996, 6: 263-71, J Am Diet Assoc 1997, 97:1110-5 and J Strength Cond Res., 2001, 15 (3): 290-5). Only a few clinical evidences as such come from the subjects of professional athlete (Forgo I, MMW Munch Med Wochenschr., 125(38):822-4, 1983) or sports teachers (Pieralisi G, et al., Clin Ther., 13(3):373-82, 1991) only. That is, ginseng root has been reported to have no effect on maximal oxygen uptake (VO₂max) and lactate threshold (LDH) of soccer players (Int J Sport Nutr. 1999 9(4): 371-7). It also has been reported not to change lactate threshold and physical performance in physically active Thai men. It means that ginseng root does not show an ergogenic effect on aerobic fitness enhancement of well-fit human (J Med Assoc Thai 2007 90(6): 1172-9). There is a report that ginseng root does not promote an anabolic hormonal status following resistance exercise (J Strength Cond Res., 2002 16 (2): 179-83). In addition, Eleutherococcus has been reported not to support an ergogenic effect regarding metabolic, performance or phychologic parameters associated with submaximal and maximal aerobic exercise tasks (Med Sci Sports Exerc. 1996, 28 (4): 482-9). It is also reported that ginseng root extracts may increase aerobic performance under appropriate conditions such as use of standardized root extract, daily dose is above 2 g, large number of subjects and long treatment period (Am J Clin Nutr., 2000, 72: 624S-36S). Accordingly, there has been no concreted research results providing the effect of ginseng relating to improvement of physical endurance performance of ordinary people as well as athlete.

Ginsenoside, a special group of triterpenoid saponins, can be classified into two sub-groups, dammarane type and oleanane type according to the skeleton of their aglycones. Ginsenosides are found specifically in Panax species and up to now more than 150 naturally occurring ginsenosides have been isolated from roots, leaves/stem, fruit or flower head. Ginsenosides have been researched in many studies since they have been recognized as main active substances showing ginseng's efficacy. Ginsenosides are important bioactive components in ginseng, and sugar chains of ginsenosides are closely related to the bioactivity. Ginseng saponins (ginsenosides) are extracted from the root and leaves of ginseng. Many studies have been focused on converting major ginsenosides to the minor ginsenoside, Rg3 which is more active. Due to the difficulty in preparing ginsenoside Rg3 and Rg2, the compounds have been mainly prepared through heating, enzymatic and strong acid treatment (Phytochemistry 2004, 65 (3): 337-44, Phytochemistry 2008, 69 (1): 218-24, Chem Pharm Bull 2003 51(4): 404-8)

Otherwise, although the supplements including the compounds such as steroid, caffeine, sodium bicarbonate, sodium citrate and the like may improve exercise performance remarkably, too much intake thereof will cause a lethal side effect and break our health after all.

Accordingly, many researches are conducting now for developing functional supplement by using a natural product with a guaranteed safety such as plant extract. For example, Korean patent No. 526164 discloses a composition for enhancing exercise performance comprising squalene and plant extract.

### BRIEF DESCRIPTION OF THE DRAWING

FIG. 1 is a graph to show the ginsenosides contents of UG0407, UG0507 and UG0712 in comparison with another ginseng extracts.

FIG. 2 is a graph to show the results of exercise performance improvement of the ginseng leaf extract powder.

FIG. 3 is a graph to show the results of exercise performance improvement of the processed ginseng leaf extract powder.

FIG. 4 is a graph to show the results of exercise performance improvement of the mixture of ginseng leaf extract and processed ginseng leaf extract powder after 2 weeks-exercise.

FIG. 5 is a graph to show the results of exercise performance improvement of the mixture of ginseng leaf extract and processed ginseng leaf extract powder after 8 weeks-exercise.

FIG. 6 is a graph to show the results of non-exercise performance improvement of the mixture of ginseng leaf extract and processed ginseng leaf extract powder after 6 weeks.

FIG. 7 is a graph to show the results of non-exercise performance improvement of the mixture of ginseng leaf extract and processed ginseng leaf extract powder after 9 weeks.

FIG. 8 is a graph to show the results of blood creatine kinase concentration of UG0507 in the exercise group.

FIG. 9 is a graph to show the results of blood creatine kinase concentration of UG0712 in the exercise group after 2 weeks.

FIG. 10 is a graph to show the results of blood creatine concentration of UG0407 in the exercise group.

FIGS. 11 and 12 are graphs to show the results of LDH (lactate dehydrogenase) concentration of UG0407 and UG0712 in blood of non-exercise group after maximal running test at 6^{th} week, respectively.

FIG. 13 is graph to show the results of LDH concentration of UG0507 in muscle of non-exercise group.

FIGS. 14 and 15 are graphs to show the results of LDH (lactate dehydrogenase) concentration of UG0407 and UG0712 in blood of exercise group, respectively.

FIGS. 16 and 17 are graphs to show the results of LDH concentration of UG0507 and UG0712 in muscle of exercise group.

FIG. 18 is a graph to show the results of blood lactic acid concentration of UG0407 in the exercise group.

FIG. 19 is a graph to show the results of blood lactic acid concentration of UG0507 in the exercise group.

FIG. 20 is a graph to show the results of lactic acid concentration of UG0712 in blood of the exercise group.

FIG. 21 is a graph to show the results of lactic acid concentration of UG0712 in blood of the non-exercise group.

FIGS. 22 and 23 are graphs to show the results of blood corticosterone level of UG0407 in the non-exercise group and the exercise group, respectively.

FIGS. 24 and 25 are graphs to show the results of blood corticosterone level of UG0507 in the non-exercise group and the exercise group, respectively.

FIG. 26 is a graph to show the results of blood corticosterone level of UG0712 in the non-exercise group.

FIG. 27 is a graph to show the results of blood corticosterone level of UG0712 in the exercise group.

FIG. 28 is a graph to show the results of CS (citrate synthase) of UG0407 in muscle of the exercise group.

FIG. 29 is a graph to show the results of CS (citrate synthase) of UG0712 in muscle of the non-exercise group.

FIG. 30 is a graph to show the results of CS (citrate synthase) of UG0712 in muscle of the exercise group.

FIG. 31 is graph to show the results of NO (nitric oxide) level of UG0407 in blood of the exercise group.

FIG. 32 is a graph to show the results of NO (nitric oxide) level of UG0507 in muscle of the exercise group.

FIG. 33 is a graph to show the results of NO (nitric oxide) level of UG0712 in blood of the non-exercise group.

FIG. 34 is a graph to show the results of NO (nitric oxide) level of UG0712 in muscle of the non-exercise group.

FIG. 35 is a graph to show the results of NO (nitric oxide) level of UG0712 in blood of the exercise group, wherein the blood was collected before 2 weeks-exercise.

FIG. 36 is a graph to show the results of NO (nitric oxide) level of UG0712 in blood of the exercise group, wherein the blood was collected after 2 weeks-exercise.

FIG. 37 is a graph to show the results of NO (nitric oxide) level of UG0712 in muscle of the exercise group.

FIG. 38 is a graph to show the results of SOD (superoxide dismutase) inhibition rate of UG0407 in muscle of the exercise group.

FIG. 39 is a graph to show the results of SOD (superoxide dismutase) inhibition rate of UG0507 in muscle of the exercise group.

FIG. 40 is a graph to show the results of SOD (superoxide dismutase) inhibition rate (%) of UG0712 in muscle of the exercise group.

FIGS. 41 and 42 are graphs to show the results of GPx (glutathione peroxidase) level of UG0407 in muscle of the non-exercise group and the exercise group, respectively.

FIG. 43 is a graph to show the results of GPx (glutathione peroxidase) level of UG0507 in liver of the exercise group.

FIG. 44 is a graph to show the results of GPx (glutathione peroxidase) level of UG0712 in liver of the exercise group.

FIG. 45 is a graph to show the results of ATPase test of UG0712 in soleus muscle.

FIG. 46 is a graph to show the results of ATPase test of UG0712 in red gastrocnemius muscle.

FIG. 47 is a graph to show the results of change of VO₂ max values of UG0712 .

FIG. 48 is a graph to show the results of change of AT values of UG0712 .

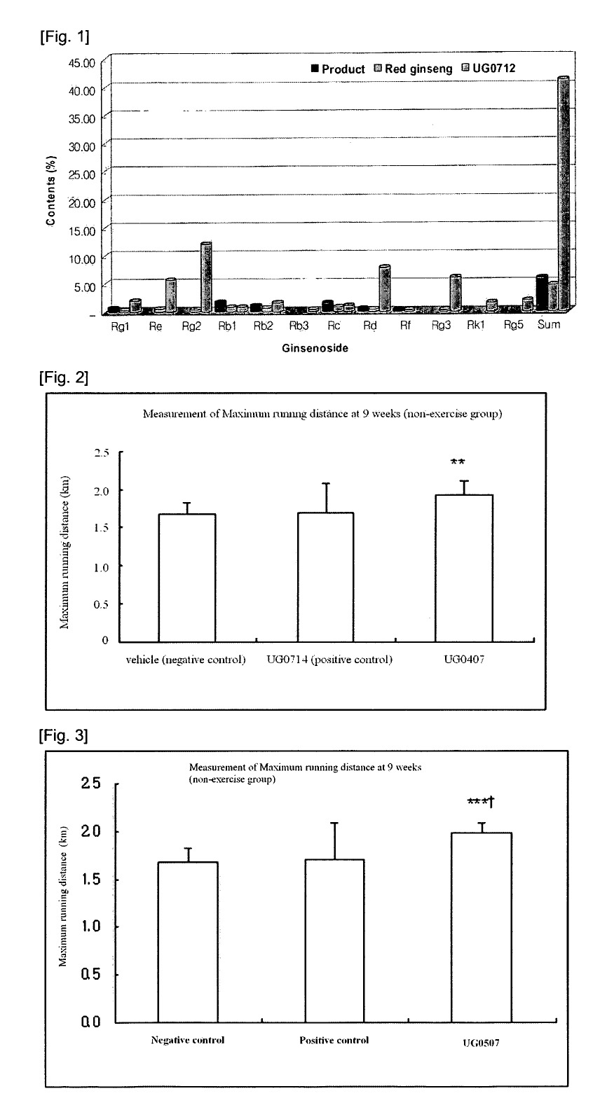

### DETAILED DESCRIPTION OF THE INVENTION

### Technical Purpose

The present invention has been invented according to the requirements as above, and thus the purpose of the present invention is to provide a composition comprising Panax species plant leaves extract or processed Panax species plant leaves extract, or mixture of the both as an active ingredient which efficiently improves exercise performance and fatigue recovery, inhibits the accumulation of fatigue markers in blood and prevents oxidation responses with no adverse effect to the subjects of ordinary people as well as athletes.

### Technical Solution

To achieve the above-mentioned purpose, the present invention provides an antioxidant composition for improving exercise performance and fatigue recovery comprising Panax species plant leaves extract or processed Panax species plant leaves extract, or mixture of the both as an active ingredient.

Preferably, the present invention provides the composition wherein the Panax species plant leaves extract, processed product of the leaves extract, or mixture of the both comprises 3-O-glycosides of protopanaxatriol and 3-O-glycosides of protopanaxadiol.

In the Panax species plant leaves extract, processed product of the leaves extract, or mixture of the both according to the present invention, the total content of ginsenosides is preferably 30 wt % or more, more preferably 40 wt % or more.

An embodiment of the present invention provides the composition for improving exercise performance or fatigue recovery, or prevention of oxidation reaction wherein the Panax species plant leaves extract, processed product of the leaves extract, or mixture of the both comprises one or more ginsenosides selected from the group consisting of Rg3, Rg5, and Rk1, as active ingredient.

In the Panax species plant leaves extract according to the present invention, the total content of Rg3, Rg5 and Rk1 is 1.5 wt % or more. In the processed Panax species plant leaves extract, or mixture of the Panax species plant leaves extract and the processed product of the leaves extract, the total content of Rg3, Rg5 and Rk1 is 5 wt % or more, preferably 10 wt % or more.

In the present invention, said Panax species plant can be selected from the group consisting of Panax ginseng, Panax japonicum, Panax quinquefolium, Panax notoginseng, Panax trifolium Panax pseudoginseng Panax vietnamensis, Panax elegatior, Panax wangianus, and Panax bipinratifidus.

In the composition according to the present invention, said Panax species plant leaves extract and processed Panax species plant leaves extract can be mixed with the content ratio of 1:0.1 to 5, preferably 1:0.1 to 3, more preferably 1:0.5 to 2, respectively.

The present composition comprising mixture of Panax species plant leaves extract and processed Panax species plant leaves extract may further contain one or more component(s) selected from the group consisting of squalene, Saururus chinensis aqueous extract, Acanthopanax sessiliflorus aqueous extract, aqueous extract of Cordycepsmilitaris and Paecilomyces japonica, cola nut powder or extract, vitamins, minerals, taurine, creatine, phosphatidylcholine, glutamine, L-arginine and L-carnitine.

Preferably, the present invention provides a method for improving exercise performance and fatigue recovery comprising administering to a subject in need thereof a composition comprised of a Panax species plant leaves extract or a processed product of the leaves extract or a mixture of the both.

Preferably, the present invention also provides a method for reducing exercise induced oxidative stress, reducing the levels of one or more fatigue markers selected from the group consisting of creatine, creatine kinase, lactate dehydrogenase(LDH), lactate, and corticosterone , or inhibiting NO (nitric oxide) or SOD (superoxide dismutase) oxidation, or enhancing GPx (glutathione peroxidase) activity, comprising administering to a subject in need thereof a composition comprised of the Panax species plant leaves extract, the processed product of the leaves extract or the mixture of the both.

Preferably, the present invention provides a method for enhancing VO₂ max, AT (anaerobic threshold) or citrate synthase activity said method comprising administering to a subject in need thereof a composition comprised of the mixture of Panax species plant leaves extract and the processed product of the leaves extract.

Preferably, the present invention provides a use of a Panax species plant leaves extract, a processed product of the leaves extract or a mixture of the both in the manufacture of a composition for improving exercise performance and fatigue recovery or reducing exercise induced oxidative stress.

Preferably, the present invention provides a use of a Panax species plant leaves extract, a processed product of the leaves extract, or a mixture of the both in the treatment of exercise induced fatigue or exercise induced oxidative stress.

### INDUSTRIAL APPLICABILITY

As well as increasing the exercise performance time, inhibiting the accumulation of fatigue markers in blood and preventing oxidation response, intake of the composition according to the present invention also improves aerobic exercise capacity according to maximum oxygen intake, i.e., cardiopulmonary exercise endurance, and thus the composition according to the present invention is useful to improve physical strength and exercise capacity, and safe to human.

### EMBODIMENT TO CARRY OUT THE INVENTION

To achieve the purpose, the present invention provides composition for improving exercise performance, fatigue recovery or prevention of oxidation response comprising mixture of Panax species plant leaves extract, processed Panax species plant leaves extract or mixture of the both as an active ingredient.

According to one embodiment of the present invention, said Panax species plant leaves extract, processed product of the leaves extract, or mixture of the both provides the composition comprising 3-O-glycosides of protopanaxatriol and 3-O-glycosides of protopanaxadiol. The content ratio of 3-O-glycosides of protopanaxatriol: 3-O-glycosides of protopanaxadiol in the Panax species plant leaves extract is preferably 1:0.1 to 1, more preferably 1:0.5 to 1. The content ratio of 3-O-glycosides of protopanaxatriol: 3-O-glycosides of protopanaxadiol in the processed Panax species plant leaves extract is 1:0.1 to 1.5, preferably 1:0.5 to 1.5, more preferably 1:0.7 to 1.5. The content ratio of 3-O-glycosides of protopanaxatriol: 3-O-glycosides of protopanaxadiol in the mixture of Panax species plant leaves extract and the processed product of the plant leaves extract is 1:0.1 to 1.5, preferably 1:0.5 to 1.5, more preferably 1:0.7 to 1.5. 3-O-glycosides of protopanaxadiol contain such ginsenosides as Rb1, Rb2, Rb3, Rc, Rd, Rg3(R,S), Rg5, Rk1 or the like. 3-O-glycosides of protopanaxatriol contain such ginsenosides as Re, Rg1, Rg2, or the like. In terms of the exercise performance and fatigue recovery effects and antioxidant effect, advantages can be obtained within aforesaid content ratios.

In one embodiment of the composition according to the present invention, each of said Panax species plant leaves extract, processed product of the leaves extract, or mixture of the both contains ginsenosides in amount of 30 wt % or more, preferably 40 wt % or more in total.

In one embodiment of the composition according to the present invention, said Panax species plant leaves extract, processed product of the leaves extract, or mixture of the both comprises one or more ginsenosides selected from the group consisting of Rg3, Rg5, and Rk1, as active ingredient.

In one embodiment of the composition according to the present invention, Panax species plant leaves extract, processed product of the leaves extract, or mixture of the both contains protopanaxadiols such as Rg3, Rg5 and Rk1 in amount of 1.5 wt % or more of the total weight amount of the composition. The processed Panax species plant leaves extract and the mixture of Panax species plant leaves extract and the processed product of the leaves extract contains protopanaxadiols such as Rg3, Rg5 and Rk1 in amount of 10 wt % or more of the total weight amount of the composition. In terms of the exercise performance and fatigue recovery effects and antioxidant effect, advantages can be obtained within aforesaid content ratios.

In one embodiment of the composition according to the present invention, Panax species plant leaves extract, the processed product of the leaves extract and the mixture of the both contain 40% or more of total ginsenosides, and 90% or more of total saponin. In particular, Panax species plant leaves extract contains 50% or more of total ginsenoside.

Table 1 is to show the comparison results of UG0712(mixture of Panax species plant leaves extract and the processed product of the leaves extract) in ginsenoside content with ginseng products. From Table 1, it can be known that the Panax species plant leaves extract of the present invention has a much higher content of ginsenosides as compared with other commercially available ginseng products.

TABLE 1
Ginsenoside content of UG0712 in comparison to marketed ginseng products

| | | Results | |
|---|---|---|---|
| | | | Total |
| Item | Company | Rg3, Rg5, Rk1 | Ginsenoside |
| UG0712 | Unigen | 10.01% | 41.05% |
| (Mixture of UG0407 and UG0507) | | | |
| UG0407 (Ginseng Leaf) | Unigen | 1.7 | 56.7 |
| UG0507 (Processed Ginseng Leaf) | Unigen | 16.4 | 43.6 |
| UG0714 (Ginseng Root) | Commodity | 0.79 | 8.04 |
| Ginseng Gold, | GNC | 0.34% | 4.97% |
| Korean white ginseng root | | | |
| Ginseng Gold, | GNC | 0.17% | 4.10% |
| Standardized American white ginseng | | | |
| American Ginseng extract | Johnson & Barana | 0.35% | 11.48% |
| Pharmaton | Boehringer | N.D | 1.7% |
| | Ingelheim | | |
| Nature's Resource ® Ginseng | Nature's Resource | 0.17% | 11.24% |
| GinSynergy | BIOGLAN | 0.09% | 6.09% |
| Ginseng Panax Integratore alimentare | BODY SPRING | 1.04% | 6.02% |
| American Ginseng PE | STAUBER | 0.41% | 10.50% |
| Panax Ginseng PE | STAUBER | 0.1% | 3.4% |
| American ginseng powder | Hsu's Ginseng | 0.35% | 8.01% |
| American Ginseng | NATUREX | 0.1% | 2.1% |
| Root PE 1% ginsenosides Q | | | |
| Ginsenipure ™ Ginseng americ | NATUREX | 0.43% | 18.43% |

The structures and physicochemical properties of protopanaxadiols such as Rg3, Rg5 and Rk1, contained in the present Panax species plant leaves extract, processed product of the leaves extract or the mixture of the both, are shown in Table 2.

TABLE 2
Structures and Physicochemical Properties of Rg3, Rg5 and Rk1

| Ginsenoside | 20(S,R)-Rg3 | | Rg5 |
|---|---|---|---|
| Name | | | |
| Ginsenoside Structure | 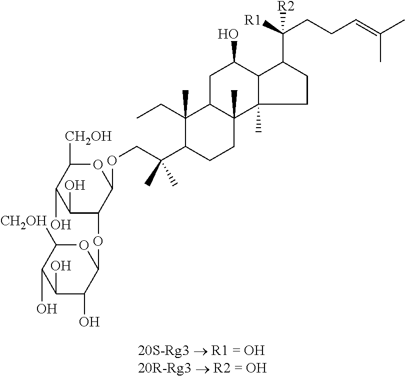 | | 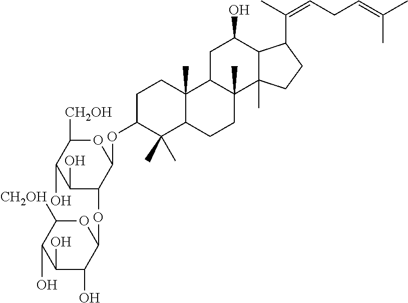 |
| Molecular | C₄₂H₇₂O₁₃ | C₄₂H₇₂O₁₃ | C₄₂H₇₀O₁₂ |
| Formular | | | |
| Molecular | 785.023 | 785.0343 | 767.0078 |
| Weight | | | |
| Appearance | White powder | White powder | White powder |
| Melting | 248~250° C. | 299~303° C. | 186~188° C. |
| Point (° C.) | | | |
| Soluble in | Alcohol | DMSO | Alcohol |

| Structures and Physicochemical Properties of Rg3, Rg5 and Rk1 | |
|---|---|
| Ginsenoside | Rk1 |
| Name | |
| Ginsenoside Structure | 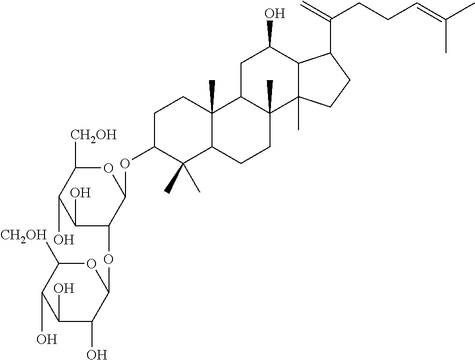 |
| Molecular | C₄₃H₇₄O₁₂ |
| Formular | |
| Molecular | 783.0504 |
| Weight | |
| Appearance | White powder |
| Melting | 178~181° C. |
| Point (° C.) | |
| Soluble in | Alcohol |

In the present invention, said Panax species plant can be Panax ginseng, Panax japonicum, Panax quinquefolium, Panax notoginseng, Panax trifolium, Panax pseudoginseng, Panax vietnamensis, Panax elegatior, Panax wangianus, Panax bipinratifidus or the like, but not limited thereto.

In one embodiment of the composition according to the present invention, said Panax species plant leaves extract and processed Panax species plant leaves extract can be mixed with the content ratio of 1:0.1 to 10, preferably 1:0.1 to 5, more preferably 1:0.1 to 3, still more preferably 1:0.5 to 2, respectively.

In one embodiment of the composition according to the present invention, the Panax species plant leaves extract, processed Panax species plant leaves extract, or mixture of the both increases the exercise performance, inhibits the accumulation of fatigue markers and prevents oxidation response, and increases aerobic exercise capacity with respect to the maximum oxygen consumption, i.e., pulmonary exercise endurance, and thus is useful to improve physical strength and exercise capacity.

In detail, the present Panax species plant leaves extract, processed Panax species plant leaves extract, or mixture of the both improves exercise capacity in animal, inhibits the accumulation of fatigue markers in muscle and/or blood, due to exercise, such as CK(creatine kinase), LDH(lactate dehydrogenase), lactate, corticosterone, improves exercise performance by increasing CS(citrate synthase) activity, prevents oxidation response by inhibiting NO(nitric oxide), inhibiting SOD(syperoxide dismutase) oxidation, and increasing GPx(glutathione peroxidase) activity, and improves exercise capacity by increasing VO₂ max and AT (Anaerobic Threshold).

In one embodiment of the composition according to the present invention, said mixture of Panax species plant leaves extract and processed Panax species plant leaves extract may be in the form of powder, but are not limited to. The powder form of the extract can be prepared by freeze-drying, hot air drying, electromagnetic wave or the like.

In one embodiment of the composition according to the present invention, the Panax species plant leaves extract can be obtained by reflux-extraction with an extract solvent selected from water, C₁₋₄ alcohol, or mixtures thereof.

In one embodiment of the composition according to the present invention, the processed Panax species plant leaves extract can be obtained by reflux-extraction with an extract solvent selected from water, C₁₋₄ alcohol, or mixtures thereof, freeze-drying the reflux-extract, processing the freeze-dried extract by adding water and glacial acetic acid thereto with stirring at 60 to 100° C. , and drying the processed extract.

In one embodiment of the composition according to the present invention, the mixture of Panax species plant leaves extract and processed product of the leaves extract is obtained by the following steps:

(a) reflux-extracting Panax species plant leaves with an extract solvent selected from water, C₁₋₄ alcohol, or mixtures thereof, and then freeze-drying the reflux-extract to obtain the Panax species plant leaves extract powder;

(b) processing the Panax species plant leaves extract powder by adding water and glacial acetic acid thereto with stirring at 60 to 100° C. , and drying the processed extract to obtain the processed product of the leaves extract powder; and

(c) mixing the Panax species plant leaves extract powder obtained from process (a) with the processed product of the leaves extract powder obtained from process (b).

The extract solvent can be water, C₁₋₄ alcohol, or mixtures thereof, and the alcohol is preferably ethanol, more preferably 70% ethanol.

In the composition according to the present invention, the mixture of Panax species plant leaves extract and processed Panax species plant leaves extract can further comprise one or more active components which have the same or similar function.

One embodiment of the composition according to the present invention can further comprise one or more components selected from the group consisting of squalene, Saururus chinensis aqueous extract, Acanthopanax sessiliflorus aqueous extract, aqueous extract of Cordycepsmilitaris and Paecilomyces japonica, amino acids or derivatives thereof, such as taurine, creatine, glutamine, L-arginine, L-carnitine, phosphatidylcholine, cola nut powder or extract, vitamins, and minerals.

Said aqueous extracts of Saururus chinensis, Acanthopanax sessiliflorus, and Cordycepsmilitaris and Paecilomyces japonica can be prepared according to conventional methods or purchased from extracts are commercially available products.

Squalene is a highly unsaturated hydrocarbon compound having 6 double bonds, and generally obtained by extracting from the shark liver oil and purifying the extract. Squalene has physiological activities such as oxygen-supply action, sterilization activity and the like. In particular, it has been known to combine with hydrogen of water and release oxygen therefrom, which is supplied to cells in the body to activate the cells.

Saururus chinensis is a perennial plant, and has various pharmacological activities. It has been known to have remarkable effects in preventing and treating adult diseases such as constipation, diabetes, liver disease, cancer, hypertension, cardiac disease, female disorders and nephropathy.

Acanthopanax sessiliflorus is in the family Araliaceae, and its a dried root and bark have been used for treating stomach disease, arthritis, lumbago, degenerative arthritis syndrome, dropsy, beriberi, bruise, swelling and the like.

Cordycepsmilitaris or Paecilomyces japonica, which is small size fungus of ascomycete family, are parasitic on an insect and produce ascocarp in dead body of the host insect. Cordycepsmilitaris and Paecilomyces japonica are known to clean up the bronchus, eliminate impurities in the blood vessel, and strengthen cardiac contractile force. It is also known as effective for cell activation and recovery, immune function improvement, blood sugar level normalization, and treatment of anemia and obesity.

Amino acids or derivatives thereof, such as taurine, creatine, glutamine, L-arginine and L-carnitine, can help recovery of muscle fatigue after exercising, and can be directly used as energy source.

Phosphatidylcholine is a compound comprising lipid, phosphorous and nitrogen, and exists abundantly in egg yolk, soy bean oil, liver, brain and the like. It is one of the major components of cell membranes, and known as an effective fatigue recovery material.

Cola nut is in the family Sterculiaceae, and represents a nut of Cola acuminate or Cola nitida containing caffeine, originated from the tropical region of Africa . It has been used as a raw material to make alcohol-free drinks and drugs, and as a herbal medicine for treating drug intoxication, hangover, and diarrhea. Cola nut can be added to the composition according to the present invention in the form of extract or powder.

Vitamins useful to the present invention include Vitamin B₁, Vitamin B₂, Vitamin B₆, nicotinic acid amide, and Vitamin C. Minerals includes MgCl₂, KCl, NaCl, Ca-lactate, ammonium iron citrate and the like which can be used in mixture.

The composition according to the present invention can be used as a composition for improving exercise performance, fatigue recovery, and inhibiting oxidation response.

In addition to the active ingredient described in the above, a pharmaceutically acceptable carrier can be further contained in the composition according to the present invention for its administration. For the pharmaceutically acceptable carriers, saline, sterile water, Ringer's solution, buffer saline, dextrose solution, maltodextrine solution, glycerol, ethanol can be used, and mixtures of two or more them also can be used. If necessary, other conventional additives such as antioxidant, buffer, bacteriostatic agent or the like can be added. Also, it can be formulated into injection dosage form such as aqueous solution, suspension, emulsion, etc., pellet, capsule, granule or tablet by further adding diluent, dispersant, surfactant, binder and lubricant. Further, it can be preferably formulated according to proper methods in this field or methods disclosed in Remington's Pharmaceutical Science (the latest ver., Mack Publishing Company, Easton Pa.), depending upon diseases or ingredients.

The composition according to the present invention can be administered parenterally [e.g., intra venous (i.v.), subcutaneous, intraperitoneal (i.p.), or topical administration] or orally according to the purpose of administration, and dose of the composition can be varied, depending on each patient's body weight, age, sex, heath condition, diet, administration period and method, excretion rate, severity of disease, and the like.

The present invention relates to a method for improving exercise performance and fatigue recovery comprising administering to a subject in need thereof a composition comprised of a Panax species plant leaves extract or a processed product of the leaves extract or a mixture of the both.

The present invention relates to a method for reducing exercise induced oxidative stress, reducing the levels of one or more fatigue markers selected from the group consisting of creatine, creatine kinase, lactate dehydrogenase(LDH), lactate, and corticosterone , or inhibiting NO (nitric oxide) or SOD (superoxide dismutase) oxidation, or enhancing GPx (glutathione peroxidase) activity, comprising administering to a subject in need thereof a composition comprised of the Panax species plant leaves extract, the processed product of the leaves extract or the mixture of the both.

The present invention relates to a method for enhancing VO₂ max, AT (anaerobic threshold) or citrate synthase activity said method comprising administering to a subject in need thereof a composition comprised of the mixture of Panax species plant leaves extract and the processed product of the leaves extract

In one embodiment of the method according to the present invention, said Panax species plant leaves extract, processed product of the leaves extract, or mixture of the both comprises 3-O-glycosides of protopanaxatriol and 3-O-glycosides of protopanaxadiol.

In one embodiment of the method according to the present invention, the ratio of 3-O-glycosides of protopanaxatriol : 3-O-glycosides of protopanaxadiol in said Panax species plant leaves extract is 1:0.1 to 1, preferably 1:0.5 to 1.

In one embodiment of the method according to the present invention, the ratio of 3-O-glycosides of protopanaxatriol: 3-O-glycosides of protopanaxadiol in said processed product of the leaves extract or said mixture of Panax species plant leaves extract and processed product of the leaves extract is 1:0.1 to 1.5, preferably 1:0.5 to 1.5, more preferably 1:0.7 to 1.5.

In one embodiment of the method according to the present invention, each of said Panax species plant leaves extract, processed product of the leaves extract, and mixture of the both contains ginsenosides in amount of 30 wt % or more in total, preferably 40 wt % or more in total.

In one embodiment of the method according to the present invention, said Panax species plant leaves extract, processed product of the leaves extract, or mixtures thereof comprise one or more ginsenoside(s) selected from the group consisting of Rg3, Rg5 and Rk1.

In one embodiment of the method according to the present invention, the Panax species plant leaves extract contains more than 1.5 wt % of Rg3, Rg5 and Rk1 in total, and the processed Panax species plant leaves extract, or mixture of Panax species plant leaves extract and processed product of the leaves extract contains more than 10 w t% of Rg3, Rg5 and Rk1 in total.

In one embodiment of the method according to the present invention, said Panax plant is selected from the group consisting of Panax ginseng, Panax japonicum, Panax quinquefolium, Panax notoginseng, Panax trifolium, Panax pseudoginseng Panax vietnamensis, Panax elegatior, Panax wangianus and Panax bipinratifidus.

In one embodiment of the method according to the present invention, the mixing ratio of said Panax species plant leaves extract: processed product of the leaves extract in the mixture is 1:0.1 to 10, preferably 1:0.1 to 5, more preferably 1:0.1 to 3, still more preferably 1:0.5 to 2.

In one embodiment of the method according to the present invention, said composition further comprising one or more components selected from the group consisting of squalene, Saururus chinensis aqueous extract, Acanthopanax sessiliflorus aqueous extract, aqueous extract of Cordycepsmilitaris and Paecilomyces japonica, cola nut powder or extract, vitamins, minerals, taurine, creatine, phosphatidylcholine, glutamine, L-arginine and L-carnitine.

The present invention relates to a use of a Panax species plant leaves extract, a processed product of the leaves extract or a mixture of the both in the manufacture of a composition for improving exercise performance and fatigue recovery or reducing exercise induced oxidative stress.

The present invention relates to a use of a Panax species plant leaves extract, a processed product of the leaves extract or a mixture of the both in the manufacture of a composition for enhancing VO₂ max, AT (anaerobic threshold) or citrate synthase activity.

The present invention relates to a use of a Panax species plant leaves extract, a processed product of the leaves extract or a mixture of the both in the manufacture of a composition for reducing the leves of one or more fatigue markers selected from the group consisting of creatine, creatine kinase, lactate dehydrogenase(LDH), lactate, and corticosterone.

The present invention relates to a use of a Panax species plant leaves extract, a processed product of the leaves extract or a mixture of the both in the manufacture of a composition for inhibiting NO (nitric oxide) or SOD (superoxide dismutase) oxidation, or enhancing GPx (glutathione peroxidase) activity.

The present invention relates to a use of a Panax species plant leaves extract, a processed product of the leaves extract, or a mixture of the both in the treatment of exercise induced fatigue or exercise induced oxidative stress.

The present invention relates to a use of a Panax species plant leaves extract, a processed product of the leaves extract, or a mixture of the both in the treatment of exercise induced fatigue by reducing the levels of one or more fatigue markers selected from the group consisting of creatine, creatine kinase, lactate dehydrogenase(LDH), lactate, and corticosterone.

The present invention relates to a use of a Panax species plant leaves extract, a processed product of the leaves extract, or a mixture of the both in the treatment of exercise induced oxidative stress by inhibiting NO (nitric oxide) or SOD (superoxide dismutase) oxidation, or enhancing GPx (glutathione peroxidase) activity.

The present invention will be explained in detail according to the following examples. However, it should be understood that the following examples are to illustrate the present invention only and the contents of the present invention are not limited to the following examples.

### EXAMPLE

### Experimental Example 1

### Preliminary Step

(1) Purchase, Quarantine and Acclimation of Animal for Test

Sprague-Dawley (SD) rats in age of 7 weeks were purchased, and all the rats were quarantined veterinarily to see their general conditions. The rats were acclimated to the experimental environment for about 7 days to select suitable and healthy rats for test. During the experiment, the test animals were bred under temperature of 22±2° C., relative humidity of 50±20%, and condition of 12 hr/day/night.

(2) Selection and Grouping of Animal for Test

To select healthy rats with no problem in exercise and have an average exercising performance, before grouping, the acclimated rats were exercised on treadmill. After removing the outlier of rats, a random grouping was made based on body weight.

(3) Identification

The breeding boxes were labeled with identification card including test number, gender, group number, individual identification number, dose, experimental period, and name of person in charge. Each rat was identified by tail marking method with oil pen.

(4) Preparation of the Test Materials

1) Preparation of Ginseng Root Extract Powder

1 kg of dried Panax ginseng root was mixed with 10 L of 70% ethanol and extracted 3 times at every 7 hrs under reflux. And the 1^{st}, 2^{nd} and 3^{rd} extracts were collected and filtered with 5 μm filter housing. The filtrate (28 L) was concentrated to 20 Brix % by vacuum evaporator under reduced pressure. The concentrate was placed in freeze-drying tray in 1 kg unit, and frozen in a deep freezer at −70° C. for 48 hours. The frozen concentrate was placed into a freeze dryer and dried for 48 hours to obtain 542 g of ginseng root extract powder (yield: 54.2%).

2) Preparation of Ginseng Leaves Extract Powder

2.5 kg of Panax ginseng leaves was mixed with 25 L of 70% ethanol and extracted for 5 hrs under reflux. And the extract was filtered with 5 μm filter housing. The filtrate (22 L) was concentrated to 15 Brix % by vacuum evaporator under reduced pressure. The concentrate was placed in freeze-drying tray in 1 kg unit, and frozen in a deep freezer at −70° C. for 48 hours. The frozen concentrate was placed into a freeze dryer (Ilshin Lab. South Korea) and dried for 48 hours to obtain 3 54 g of ginseng leaves extract powder (yield: 14.16%).

3) Preparation of Processed Ginseng Leaves Extract Powder

100 g of Ginseng leaves extract powder obtained in the above step 2) was mixed with 360 to 380 mL and 20 to 40 mL of glacial acetic acid (5 to 10%) in round bottom flask (2 L). The mixture was heated at 60 to 100° C. for 2 to 6 hours with stirring. The extract (400 mL) was concentrated to 20 Brix % by vacuum evaporator under reduced pressure. The concentrate was placed in freeze-drying tray and frozen in a deep freezer at −70° C. for 48 hours. The frozen concentrate was placed into a freeze dryer and dried for 48 hours to obtain 92.5 g of processed ginseng leaves extract (yield: 92.5%).

4) Preparation of Mixture of Ginseng Leaves Extract and Processed Ginseng Leaves Extract

350 g of ginseng leaves extract obtained in the above step 2) and 650 g of processed ginseng leaves extract obtained in the above step 3) were mixed with ribbon blender for 20 min to obtain 990 g of mixture (yield: 99%).

The doses of the test materials are shown in Table 3. 0.5% Tween 20 solution was used as a negative control group; the ginseng root extract powder obtained from the above step 1) was dissolved in 0.5% Tween 20 with sonication and used as a positive control group, and the ginseng leaves extract, the processed ginseng leaves extract, and the mixture of the both powder obtained in the above steps 2) to 4) were dissolved in 0.5% Tween 20 and used as test group 1(UG0407), test group 2(UG0507) and test group 3(UG0712), respectively.

TABLE 3
Test materials

| Group | dose (mg/kg) |
|---|---|
| Negative control group (vehicle) | — |
| Positive control group (UG0714) | 25 |
| Test group 1 (UG0407) | 25 |
| Test group 2 (UG0507) | 25 |
| Test group 3 (UG0712) | 25 |

(5) Content Analysis

For analyzing the extract powders obtained from the above steps 1) to 4), HITACHI HPLC system (pump: L-7100, detector: L-7455, interface: D-7000, column oven: L-7300, autosampler: L-7200) was used under the conditions as follows:

Stationary phase: Capcell PAK C18(5 μm), 3.0*75 mm

Mobile phase: Gradient condition with solvent A (acetonitrile) and solvent B (water)

Flow rate: 0.5 mL/min

Total analysis time: 110 min

Column over temperature: set to 40° C.

Injection amount: 10 μl per sample

Detection: at 203 nm with UV detector

Ginsenosides Rb1, Rb2, Rb3, Rc, Rd, Re and Rg1 were isolated within 60 min., and Rg2, Rg3, Rg5 and Rk1 were isolated after 70 min. The freeze-dried ginseng powder prepared according to the present method was dissolved in methanol with 2 mg/mL concentration to prepare a sample to be analyzed. Standard sample of ginsenoside was prepared with 0.2 mg/mL concentration. The analysis results are shown in Table 4.

TABLE 4
Ginsenoside contents (%)

| | Rb1 | Rb2 | Rb3 | Rc | Rd | Rc | Rg1 | Rg2 | Rg3(R,S) | Rg5 | Rk1 |
|---|---|---|---|---|---|---|---|---|---|---|---|
| UG0714 | 1.25 | 0.65 | 0.17 | 1.35 | 1.08 | 1.82 | 0.93 | ND | 0.34 | 0.36 | 0.09 |
| UG0407 | 1.3 | 2 | 1 | 3.6 | 14.1 | 19.7 | 8 | 5.3 | 1 | 0.4 | 0.3 |
| UG0507 | 2.5 | 1.1 | 0 | 0.7 | 3.4 | 0 | 0 | 19.5 | 9.4 | 4.1 | 2.9 |
| UG0712 | 0.8 | 1.6 | 0.5 | 1.1 | 7.8 | 5.7 | 2.1 | 12.1 | 6 | 2.4 | 1.8 |

As shown in Table 4, the contents of Rg3, Rg5 and Rk1 in total in the ginseng leaves extract, the processed ginseng leaves extract and the mixture of the both are 2 to 20 times or more higher than those in the ginseng root.

(6) Administration

From the day after the grouping, the test animals were orally administered with the test materials once per day with the zonde for 8 weeks for exercise group, and for 9 weeks for resting (non-exercise) group.

(7) Exercise and Non-exercise Groups

To assess effects of exercise performance, anti-fatigue after exercise, and anti-oxidant, the negative control group (vehicle, 0.5% Tween 20), positive control group (UG0714), and the test materials, i.e., test material 1(UG0407, ginseng leaves extract), test material 2 (UG0507, processed ginseng leaves extract) and test material 3 (UG0712, mixture of ginseng leaves extract and processed ginseng leaves extract) were administered to the exercise group for 8 weeks and to the non-exercise group for 9 weeks. The exercise group was adapted to exercise with treadmill more and more over the test period, and the maximum running distances were measured at 2nd week, and 8^{th} week after the start of administration. Meanwhile, the non-exercise group was adapted to exercise for 5 days before each measurement, and the maximum running distances were measured at 6^{th} week and 9^{th} week after the start of administration.

(8) General Symptom Observation and Body Weight Measurement

The general symptoms were observed 1 time/day in everyday during the test material administration period, and during the observation period, it was checked once per day whether the rat died or not. The body weights of the tested rats were measured at the grouping, just before the test material administration, every week after the start of the administration, and just before autopsy.

(9) Blood and Muscle Sampling in Autopsy

In autopsy, the whole blood was collected through the abdominal part of the rat, and divided for the analysis of anti-fatigue markers, lactic acid in blood, and corticosteroids. Each analysis was conducted within 4 hours. Muscle samples were buffered in isopentane, and frozen with liquid nitrogen to minimize the muscle damage. The frozen muscle samples were kept in deep freezer.

### Example 1

### Exercise Performance Improvement Effect

(1) Methods

1) Administration of the Test Samples

The effect of energy boosting was evaluated by measuring the exercise performance in the treadmill, and the test materials, i.e., negative control (0.5% Tween 20), UG0714 (ginseng root extract, positive control), and test materials 1 to 3(UG0407, UG0507 and UG0712) were administered to the rats.

2) Measurement

A. General symptoms observation: the general symptoms were observed 1 time/day in everyday during the period of test material administration, and during the observation period, it was checked once per day whether the rat died or not.

B. Body weight measurement: The body weights of rats were measured at the grouping, just before the test material administration, and every week after the start of the administration.

C. Exercising and Measuring maximum exercise capacity of non-exercise group: The test materials were administered to the rats in the non-exercise group (n=10) for 9 weeks, and the maximum exercise capacities of the rats were measured at 6^{th} week, and 9^{th} week. The exercise was performed on the treadmill with increasing the inclination from 0% to 15%, the speed from 20 to 40 cm/sec and the exercise duration from 10 to 20 min over 4 days, and the maximum running time was measured at 5^{th} day after the start of the exercising. Among 10 results of individual rats, the lowest and the 2^{nd} lowest results were removed and the higher 8 results were used for the exercise performance.

D. Exercising and Measuring the maximum exercise performance of exercise group: For the rats in the exercise group (n=9), the exercise was performed on the treadmill with increasing the inclination from 0% to 15%, the speed from 20 to 30 cm/sec, and the exercise duration from 30 to 40 min over the first 4 weeks. In the next 4 weeks, the exercise was performed with the inclination of 15%, the speed from 30 to 40 cm/sec, and the exercise duration from 30 to 40 min. The exercise was continued with cycle of 2 days-exercise and then 2 days-rest. Among 9 results of individual rats, the lowest and the 2^{nd} lowest results were removed and the high 7 results were used for the exercise performance.

(2) Results

1) UG0407

From the results of measurements of the maximum running distances of rats in the non-exercise group after 9 weeks' exercise with 10% inclination, 35cm/sec, and the electronic stimulation inducement for 90 min., as shown in FIG. 2, it can be known that the exercise performance of rats administered with the ginseng leaves extract powder (UG0407) statistically increased as compared with negative control (p<0.01). Also, the exercise performance of rats administered with UG0407 significantly increased as compared with the rats administered with ginseng root extract powder (UG0714, positive control group).

Therefore, it was confirmed that the administration of UG0407 improves the exercise performance of animal, compared with ginseng or negative control group.

2) UG0507

From the results of measurements of the maximum running distances of rats in the non-exercise group after 9 weeks' exercise with 10% inclination, 35cm/sec, and the electronic stimulation inducement for 90 min., as shown in FIG. 3, it can be known that the exercise performance of rats administered with the processed ginseng leaves extract powder (UG0507) statistically increased as compared with negative control (p<0.0005). Also, the exercise performance of rats administered with UG0507 statistically increased as compared with the rats administered with ginseng root extract powder (UG0714, positive control group, p<0.05).

Therefore, it can be known that the administration of UG0507 improves the exercise performance of animal, compared with ginseng or negative control group.

3) UG0712

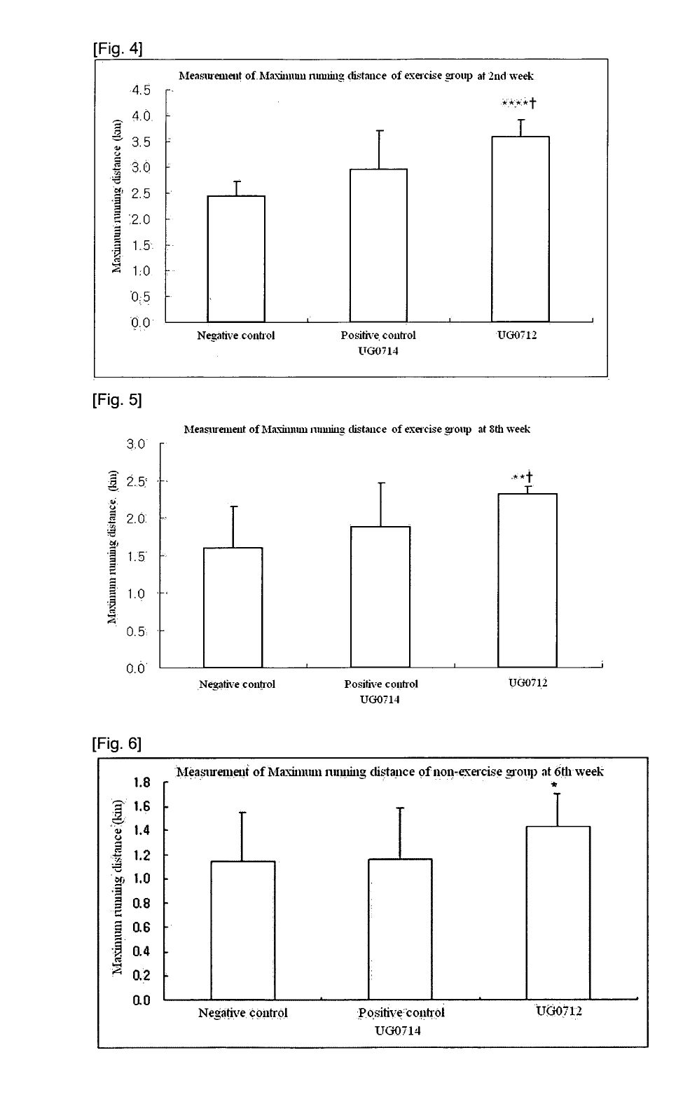

From the results of measurements of the maximum running distances of rats in the exercise group after 2 weeks' exercise with 5% inclination, 30cm/sec, and the electronic stimulation inducement for 90 min., as shown in FIG. 4, it can be known that the exercise performance of rats administered with the mixture of ginseng leaves extract and processed ginseng leaves extract powder (UG0712) statistically increased as compared with negative control (p<0.00001). Also, the exercise performance of rats administered with UG0712 significantly increased as compared with the rats administered with ginseng root extract powder (UG0714, positive control group, p<0.05).

From the results of measurements of the maximum running distances of rats in the exercise group after 8 weeks' exercise with 15% inclination, 35cm/sec, and the electronic stimulation inducement for 90 min., as shown in FIG. 5, it can be known that the exercise performance of rats administered with the mixture of ginseng leaves extract and processed ginseng leaves extract powder (UG0712) statistically increased as compared with negative control (p<0.01). Also, the exercise performance of rats administered with UG0712 significantly increased as compared with the rats administered with ginseng root extract powder (UG0714, positive control group, p<0.005).

From the results of measurements of the maximum running distances of rats in the non-exercise group after 6 weeks with 5% inclination, 35cm/sec, and the electronic stimulation inducement for 90 min., as shown in FIG. 6, it can be known that the exercise performance of rats administered with the mixture of ginseng leaves extract and processed ginseng leaves extract powder (UG0712) statistically increased as compared with negative control (p<0.05). Also, the exercise performance of rats administered with UG0712 significantly increased as compared with the rats administered with ginseng root extract powder (UG0714, positive control group, p<0.05).

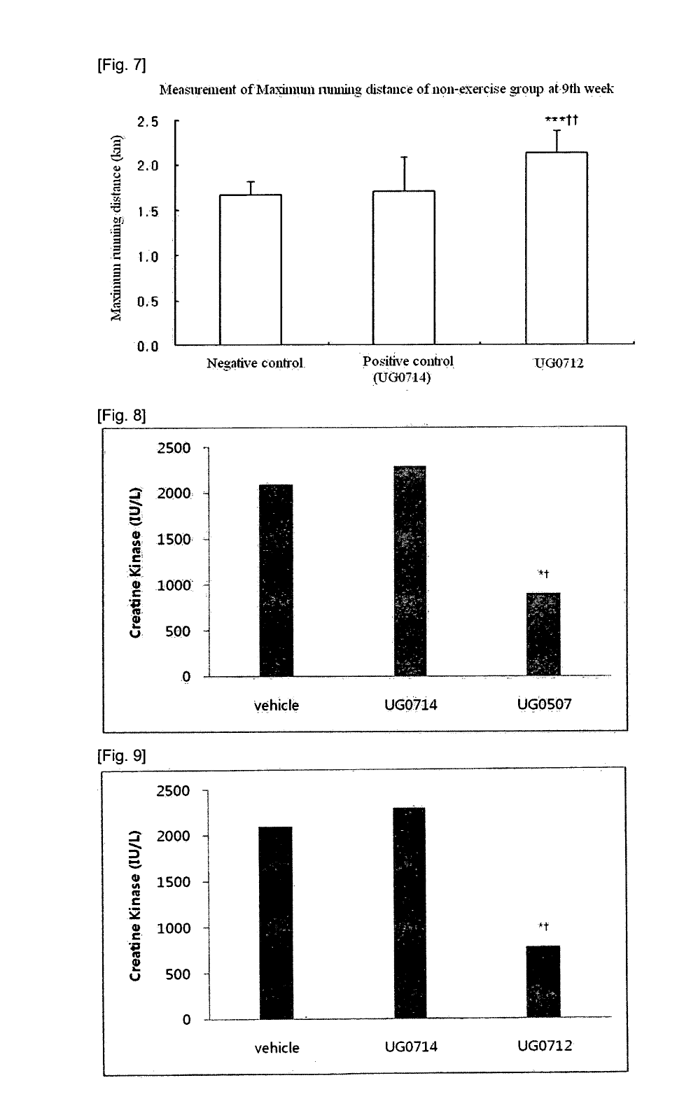

From the results of measurements of the maximum running distances of rats in the non-exercise group after 9 weeks with 10% inclination, 35cm/sec, and the electronic stimulation inducement for 90 min., as shown in FIG. 7, it can be known that the exercise performance of rats administered with the mixture of ginseng leaves extract and processed ginseng leaves extract powder (UG0712) statistically increased as compared with negative control (p<0.001). Also, the exercise performance of rats administered with UG0712 significantly increased as compared with the rats administered with ginseng root extract powder (UG0714, positive control group, p<0.05).

Therefore, it was confirmed that the administration of UG0712 improves the exercise performance of animal, compared with ginseng root extract or negative control group.

### Example 2

### Measurement of Anti-fatigue markers

To investigate anti-stress effects of the test materials to exercise stress by measuring maximum running distance of long-term and exhaustive exercise, anti-fatigue markers in blood were measured before and after the maximum running distance measurement in both exercise and non-exercise groups. For this purpose, blood samples were collected from jugular vein on 1 day before the maximal exercise test and within 20 min after exercising. Creatine kinase (CK) and LDH (lactate dehydrogenase) were measured using a biochemical blood analyzer (Hitachi 7080, Japan). Creatine was measured by using QuantiChrom, Creatine assay kit (DICT-500). Absorbance of LDH relating to anaerobic oxidation capacity was measured by using a spectrophotometer at 37° C., and all measured values are represented in unit of Umol/min/g.

Also, lactic acid and corticosteroid in blood were measured after 8^{th} week's maximum running in the exercise group, and 9^{th} week's maximum running in the non-exercise group, by using AssayMax Corticosterone ELISA Kit (Gentaur, catalog No. EC3001-1). The measured results are shown in the following Tables 5 to 20.

TABLE 5
Creatine kinase (CK) in blood of exercise group

| | |
|---|---|
| | After 2 weeks exercising |
| Exercise group | CK(IU/L) |

| | | |
|---|---|---|
| Negative control | 2091 | 955.59 |
| UG0714 | 2288 | 1267.09 |
| UG0507 | 895 | 463.91 |
| UG0712 | 774 | 347.70 |

Creatine kinase is an enzyme expressed in various tissue types. It consumes adenosing triphosphate (ATP) to catalyse the conversion of creatine to phosphocreatine and adenosine diphosphate (ADP). Clinically, creatine kinase in blood can be used as a marker of myocardial infarction, rhabdomyolysis (severe muscle breakdown), muscular dystrophy and acute renal failure.

The creatine kinase level significantly decreased in the group administered with UG0507 or UG0712 (FIGS. 8 and 9), from which it can be known that muscle injuries or the like caused by exercise could be effectively prevented by administering UG0507 or UG0712.

TABLE 6
Creatine in blood of exercise group at 10^{th} weeks

CRE (mg/dL)

| | mean | SD |
|---|---|---|
| vehicle | 0.5556 | 0.07 |
| UG0714 | 0.5857 | 0.07 |
| UG0407 | 0.4429 | 0.13 |
| vehicle: UG0714 | 0.205854795 | |
| vehicle: UG0407 | 0.032940675 | |
| UG0714: UG0407 | 0.013806565 | |

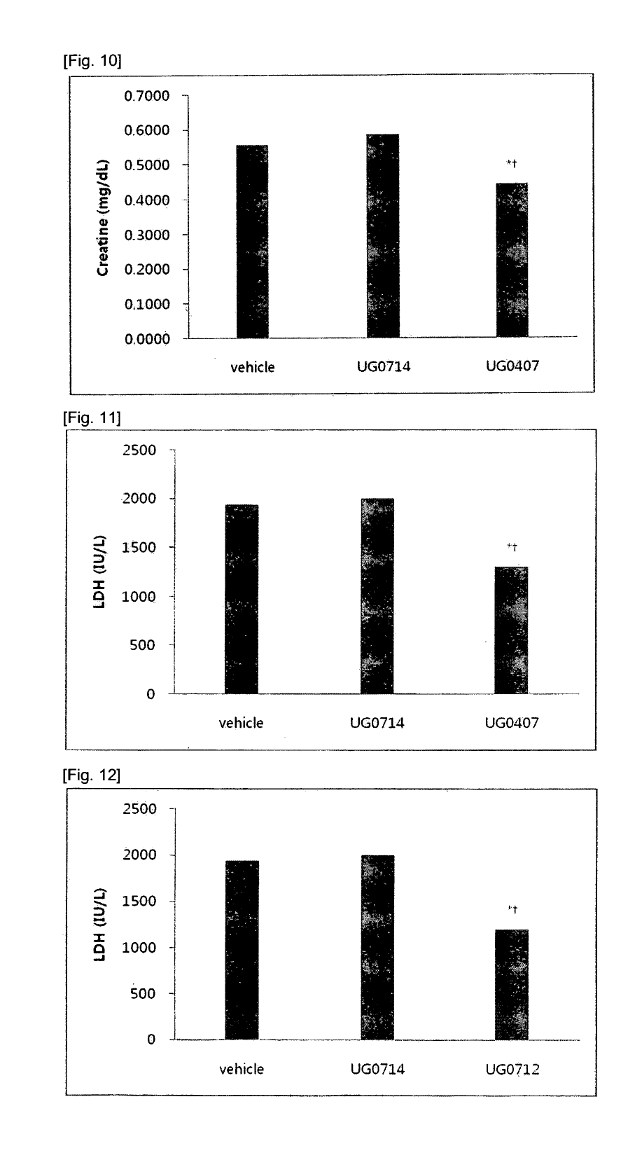

Creatine is one of fatigue makers and present as creatine phosphate in muscle. In condition of lack of oxygen, it phosphorylates ADP to ATP, and breaks down into creatine and phosphate. The creatine level increases when exercising vigorously. The creatine level decreased in the group administered with UG0407, from which it can be known the accumulation of fatigue maker due to exercising can be decreased by administering UG0407 (FIG. 10).

TABLE 7
LDH in blood of non-exercise group after
maximal running test at 6^{th} week

LDH(IU/L)

| | mean | SD |
|---|---|---|
| Negative control | 1935 | 343.45 |
| UG0714 | 1999 | 281.73 |
| UG0407 | 1305 | 210.84 |
| UG0712 | 1204 | 371.65 |

TABLE 8
LDH in muscle of non-exercise group

soleus LDH (IU/L)

| | mean | SD |
|---|---|---|
| Negative control | 1848 | 407.16 |
| Positive control (UG0714) | 1868 | 726.56 |
| UG0507 | 955 | 416.60 |

TABLE 9
LDH in blood of exercise group after
maximal running test at 8^{th} week

LDH (1^{st})

| | mean | SD |
|---|---|---|
| Negative control | 5467 | 309.85 |
| UG0714 | 4930 | 429.67 |
| UG0407 | 4559 | 403.78 |
| UG0712 | 2642 | 1100.68 |

TABLE 10
LDH in muscle of exercise group

Soleus LDH (IU/L)

| | mean | SD |
|---|---|---|
| Negative control | 6030 | 1064.64 |
| UG0714 | 5373 | 528.98 |
| UG0507 | 3916 | 588.08 |
| UG0712 | 3777 | 483.31 |

LDH is an enzyme involved in catalytic reaction between glycolytic enzyme pyruvate and lactate and present in cytoplasm. In general, fatigue after exercising is caused by excessive accumulation of lactic acid generated by energy production necessary in muscle action via the anaerobic energy system, in case of continuous and strong muscle contraction for a long time and the resultant insufficient oxygen supply into muscle cells. LDH is a good marker in the glycolytic process.

1) UG0407

When UG0407 was administered to the exercise group, LDH activity in blood decreases significantly as compared with the negative control groups and the positive control group (FIG. 14). From the results, it can be known that LDH in the exercise group generally increased as compared with those in the non-exercise group. Such results appear to be from the increase of LDH enzyme activity according to load muscle increase by regular exercising.

LDH activity in the exercise group decreased significantly when UG0407 was administered. From the results, it is expected that the administration of UG0407 helps the improvement of exercise performance by inhibiting generation of lactic acid in muscle and reducing fatigue extent.

2) UG0507

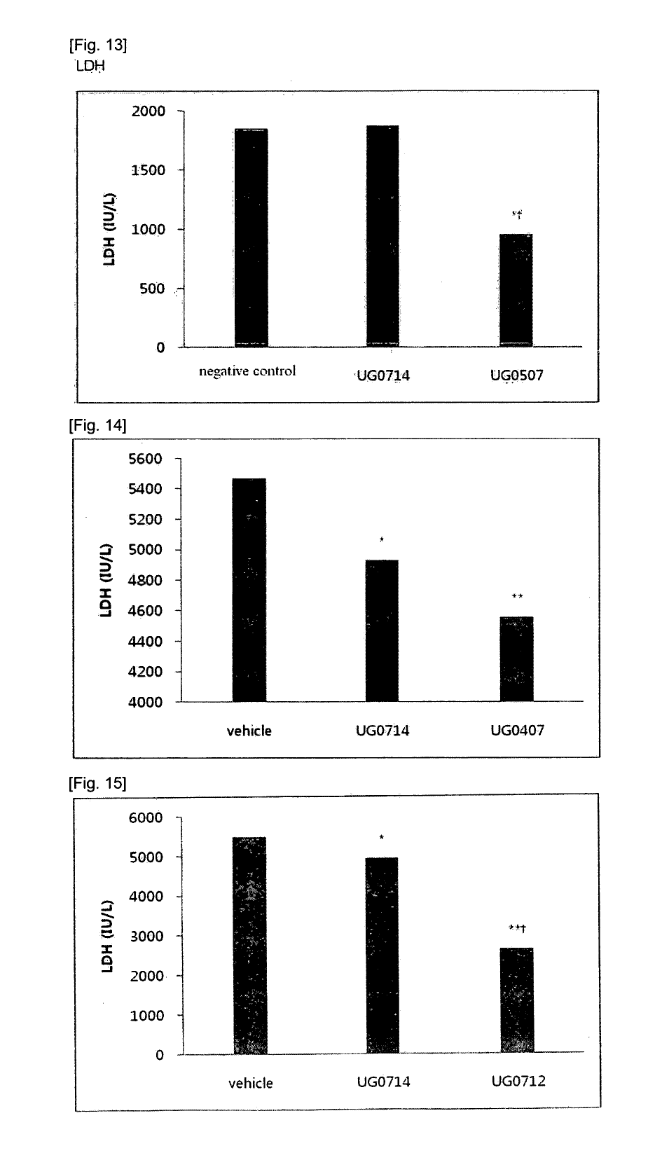

When UG0507 was administered to the non-exercise group, LDH activity in muscle decreases statistically as compared with the negative control groups and the positive control group (FIG. 13). When UG0507 was administered to the exercise group, LDH activity in muscle decreases significantly as compared with the negative control groups and the positive control group (FIG. 16). From the results, it can be known that LDH in the exercise group generally increased as compared with those in the non-exercise group. Such results appear to be from the increase of LDH enzyme activity according to load muscle increase by regular exercising.

LDH activity in the exercise group decreased significantly when UG0507 was administered. From the results, it is expected that the administration of UG0507 helps the improvement of exercise performance by inhibiting generation of lactic acid in muscle and reducing fatigue extent.

3) UG0712

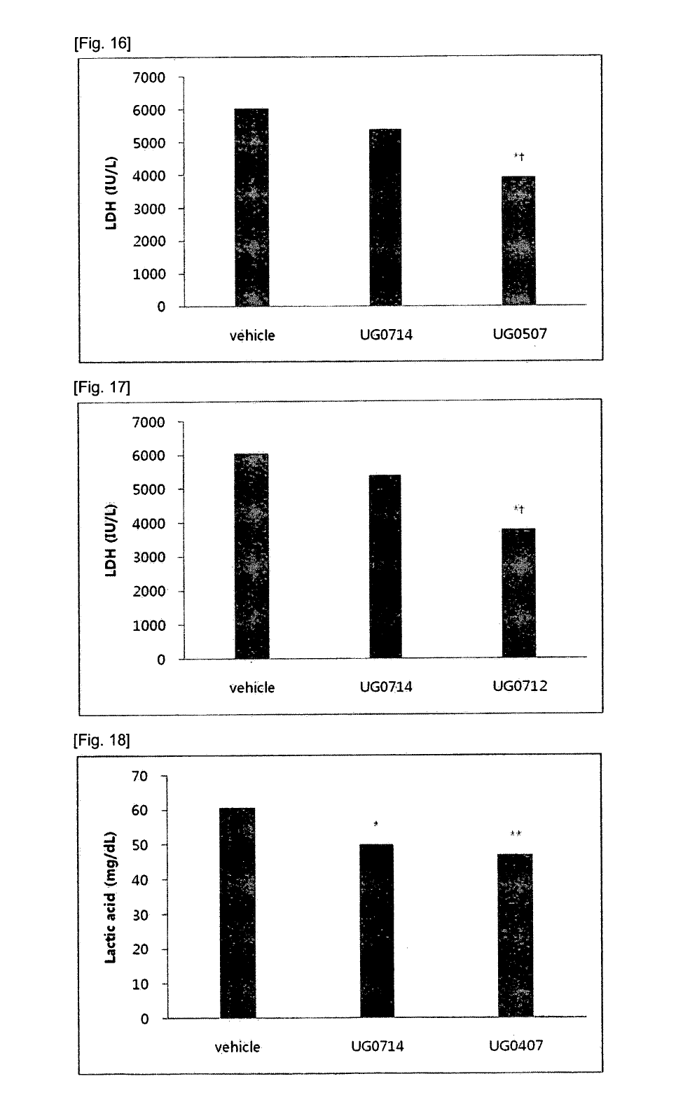

When UG0712 was administered to the non-exercise group, LDH activity in blood decreases statistically as compared with the negative control group and the positive control group (FIG. 12). When UG0712 was administered to the exercise group, LDH activities in blood and muscle decrease significantly as compared with the negative control group and the positive control group (FIGS. 15 and 17).

From the results, it can be known that LDH in the exercise group generally increased as compared with those in the non-exercise group. Such results appear to be from the increase of LDH enzyme activity according to load muscle increase by regular exercising.

LDH activity in the exercise group decreased significantly when UG0712 was administered. From the results, it is expected that the administration of UG0712 helps the improvement of exercise performance by inhibiting generation of lactic acid in muscle and reducing fatigue extent.

TABLE 11
Lactic acid in blood of exercise group

Lactic acid (mg/dL)

| | mean | SD |
|---|---|---|
| vehicle | 61 | 4.04 |
| UG0714 | 50 | 11.46 |
| UG0407 | 47 | 7.36 |
| vehicle: UG0714 | 0.0245474 | |
| vehicle: UG0407 | 0.0020805 | |
| UG0714: UG0407 | 0.2939989 | |

TABLE 12
Lactic acid in blood of exercise group

Lactic acid(mg/dL)

| | mean | SD |
|---|---|---|
| Negative control | 61 | 4.04 |
| UG0714 | 50 | 11.46 |
| UG0507 | 48 | 2.21 |

TABLE 13
Lactic acid in blood of exercise group

lactic acid(mg/dL)

| | mean | SD |
|---|---|---|
| Negative control | 61 | 4.04 |
| UG0714 | 50 | 11.46 |
| UG0712 | 42 | 4.48 |
| Negative control: UG0714 | 0.024547367 | |
| Negative control: UG0712 | 0.0000001 | |
| UG0714: UG0712 | 0.061689373 | |

TABLE 14
Lactic acid in blood of non-exercise group

Lactic acid (mg/dL)

| W. No | mean | SD |
|---|---|---|
| Negative control | 59.48 | 13.29 |
| UG0714 | 56.92 | 11.56 |
| UG0712 | 48.00 | 13.73 |
| Negative control: UG0714 | 0.3496582 | |
| Negative control: UG0712 | 0.0409178 | |
| UG0714: UG0712 | 0.0945581 | |

Lactic acid, known as one of major fatigue markers closely relating to the exercise strength and duration, is an end mediate of anaerobic glycolytic response produced from pyruvate via reduction reaction. Its level increases by intensive exercise stress, and if lactic acid is accumulated, body acidification is caused and various factors in connection with glucogenesis are inhibited.

1) UG0407

From the results, it can be known that lactic acid level in the UG0407 treatment group decreased statistically, as compared with the negative control group (FIG. 18), and accordingly, the exercise performance can be improved by administering UG0407 to decrease the fatigue factor generated from exercise. These results suggested that the fatigue factor produced by exercising decreases and thus the exercise performance can be improved by administering UG0407.

2) UG0507

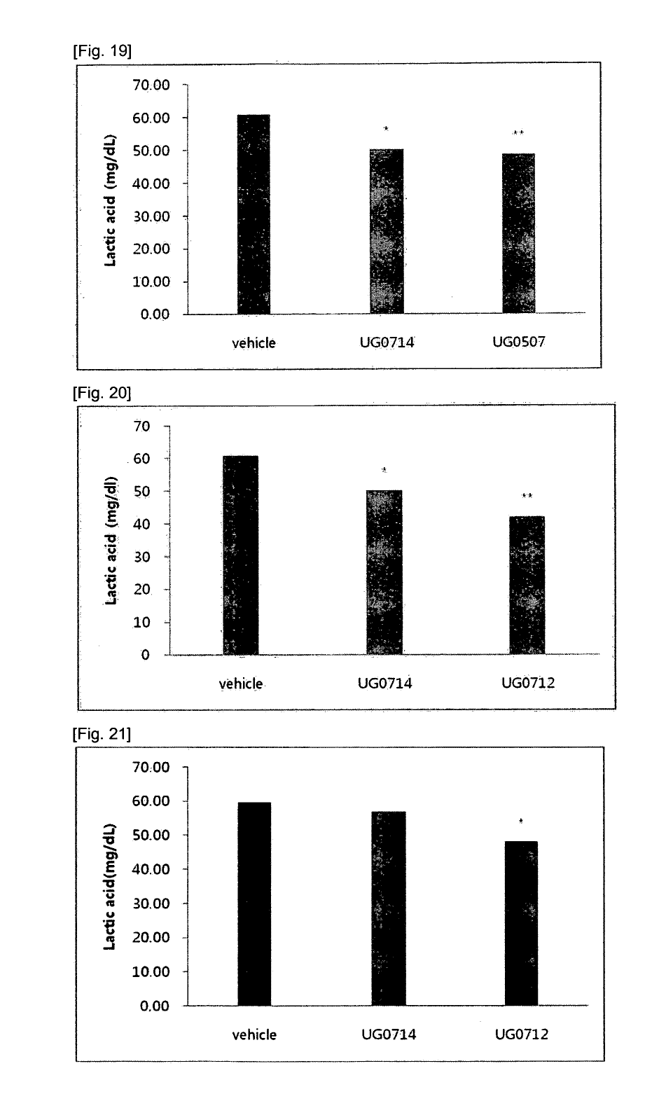

From the results, it can be known that lactic acid level in the UG0507 treatment group decreased statistically, as compared with the negative control group (FIG. 19), and accordingly, the exercise performance can be improved by administering UG0507 to decrease the fatigue factor generated from exercise. These results suggested that the fatigue factor produced by exercising decreases and thus the exercise performance can be improved by administering UG0507.

3) UG0712

From the results, it can be known that lactic acid level in the UG0712 treatment group decreased statistically, as compared with the negative control group (FIGS. 20 and 21), and accordingly, the exercise performance can be improved by administering UG0712 to decrease the fatigue factor generated from exercise. These results suggested that the fatigue factor produced by exercising decreases and thus the exercise performance can be improved by administering UG0712.

TABLE 15
Corticosterone in blood of non-exercise group

Corticosterone (ng/mL)

| | mean | SD |
|---|---|---|
| vehicle | 453 | 134.02 |
| UG0714 | 221 | 77.38 |
| UG0407 | 201 | 47.60 |
| vehicle: UG0714 | 0.0016067 | |
| vehicle: UG0407 | 0.0009584 | |
| UG0714: UG0407 | 0.2907385 | |

TABLE 16
Corticosterone in blood of exercise group

Corticosterone (ng/mL)

| | mean | SD |
|---|---|---|
| vehicle | 231 | 108.45 |
| UG0714 | 182 | 80.56 |
| UG0407 | 111 | 55.69 |
| vehicle: UG0714 | 0.17638832 | |
| vehicle: UG0407 | 0.01048202 | |
| UG0714: UG0407 | 0.05423817 | |

TABLE 17
Corticosterone in blood of non-exercise group

corticosterone(ng/mL)

| | mean | SD |
|---|---|---|
| Negative control | 453 | 134.02 |
| Positive control(UG0714) | 221 | 77.38 |
| UG0507 | 206 | 63.81 |

TABLE 18
Corticosterone in blood of exercise group

cotricosterone (ng/mL)

| | mean | SD |
|---|---|---|
| Negative control | 231 | 108.45 |
| UG0714 | 182 | 80.56 |
| UG0507 | 126 | 53.76 |

TABLE 19
Corticosterone in blood of non-exercise group

corticosterone (ng/mL)

| W. No | mean | SD |
|---|---|---|
| Negative control | 453 | 134.02 |
| UG0714 | 221 | 77.38 |
| UG0712 | 221 | 89.13 |
| Negative control: UG0714 | 0.0016067 | |
| Negative control: UG0712 | 0.0014621 | |
| UG0714: UG0712 | 0.4970829 | |

TABLE 20
Corticosterone in blood of exercise group

corticosterone (ng/mL)

| | mean | SD |
|---|---|---|
| Negative control | 231 | 108.45 |
| UG0714 | 182 | 80.56 |
| UG0712 | 134 | 39.33 |
| Negative control: UG0714 | 0.1763883 | |
| Negative control: UG0712 | 0.0221434 | |
| UG0714: UG0712 | 0.1155566 | |

Corticosteroids, known as a representative stress factor, play an important role in glycolytic process during exercising, and blood level thereof depends on the exercise strength. The blood corticosteroid level shows tendency of increase during both endurance exercise and high intensity exercise. Differently from catecholamine, the corticosteroid in blood does not decrease immediately after exercising and maintains increased level for a considerable time. If a high corticosteroid level is maintained for a long time, proteins in body are decomposed or denatured, and adverse effect inhibiting nitrogen balance can be caused.

1) UG0407

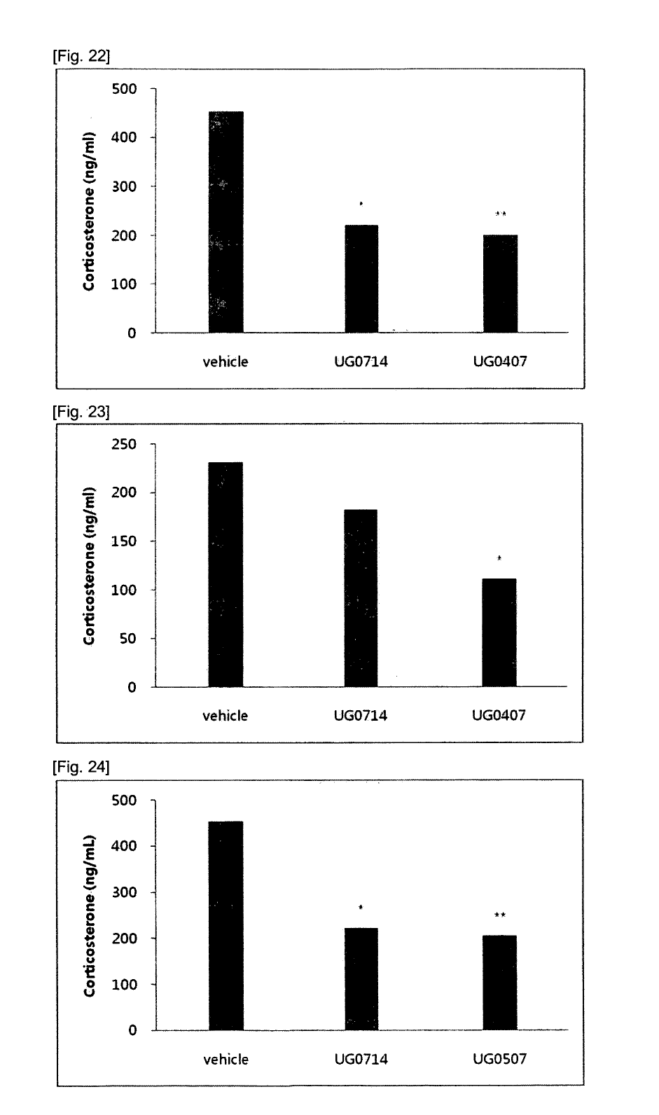

In the results, in case that UG0407 was administered to the exercise group and the non-exercise group, blood corticosterone level decreased statistically (FIGS. 22 and 23). Accordingly, it can be known that UG0407 administration can improve exercise performance more by reducing concentration of stress factors.

2) UG0507

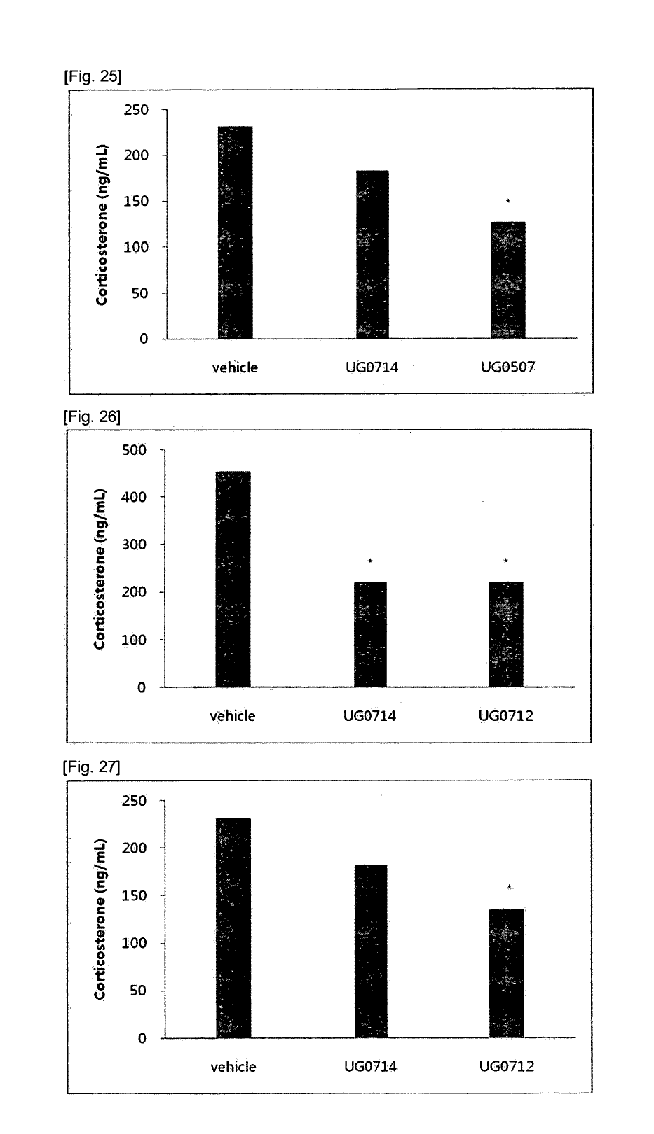

In the results, in case that UG0507 was administered to the non-exercise group and the exercise group, blood corticosterone level decreased statistically (FIGS. 24 and 25). Accordingly, it can be known that UG0507 administration can improve exercise performance more by reducing concentration of stress factors.

3) UG0712

In the results, in case that UG0712 was administered to the non-exercise group and the exercise group, blood corticosterone level decreased significantly (FIGS. 26 and 27). Accordingly, it can be known that UG0712 administration can improve exercise performance more by reducing concentration of stress factors.

### Example 3

### Measurement of Exercise Performance Improvement Effect

Muscle metabolism in connection with exercise generally goes forward to changes of increasing oxidative activity and delaying muscle fatigue state. Such changes are reflected to the activity of mito-oxidative enzymes in muscle, and depend on the exercise period and strength. The mito-oxidative enzymes include CS (citrate synthase), Cytochrome C oxidase, succinate dehydrogenase and the like. In particular, CS is known as a good marker of aerobic oxidative activity. To investigate bio-chemical markers relating to the improvement of exercise performance in both exercise and non-exercise groups, CS activity was measured by using muscle samples.

Muscle sample was added to 2 mM MgCl₂ and 2 mM EDTA solution in 50 mL TRIS, and homogenized at 4° C. The absorbance of CS (citrate synthase) relating to energy generation by aerobic oxidation in muscle was measured by spectrophotometer at 37° C. and all the measured values are represented in Umol/min/g.

TABLE 21
Citrate synthase activity in muscle of exercise group

CS activity (micromole/ml/min) in Soleus

| | mean | SD |
|---|---|---|
| vehicle | 1015 | 329.03 |
| UG0714 | 853 | 319.48 |
| UG0407 | 1300 | 317.11 |
| vehicle: UG0714 | 0.1861115 | |
| vehicle: UG0407 | 0.0774914 | |
| UG0714: UG0407 | 0.0231564 | |

TABLE 22
Citrate synthase activity in muscle of non-exercise group

CS activity(micromole/ml/min)
red gastrocnemius

| W. No | mean | SD |
|---|---|---|
| Negative control | 520 | 115.58 |
| UG0714 | 662 | 196.82 |
| UG0712 | 708 | 204.23 |
| Negative control: UG0714 | 0.0627109 | |
| Negative control: UG0712 | 0.0293347 | |
| UG0714: UG0712 | 0.3388298 | |

TABLE 23
Citrate synthase activity in muscle of exercise group

CS activity(micromole/ml/min)
White gastrocnemius

| | Mean | SD |
|---|---|---|
| Negative control | 871 | 272.21 |
| UG0714 | 790 | 119.21 |
| UG0712 | 1167 | 315.02 |
| Negative control: UG0714 | 0.2317145 | |
| Negative control: UG0712 | 0.0478116 | |
| UG0714: UG0712 | 0.0157116 | |

1) UG0407

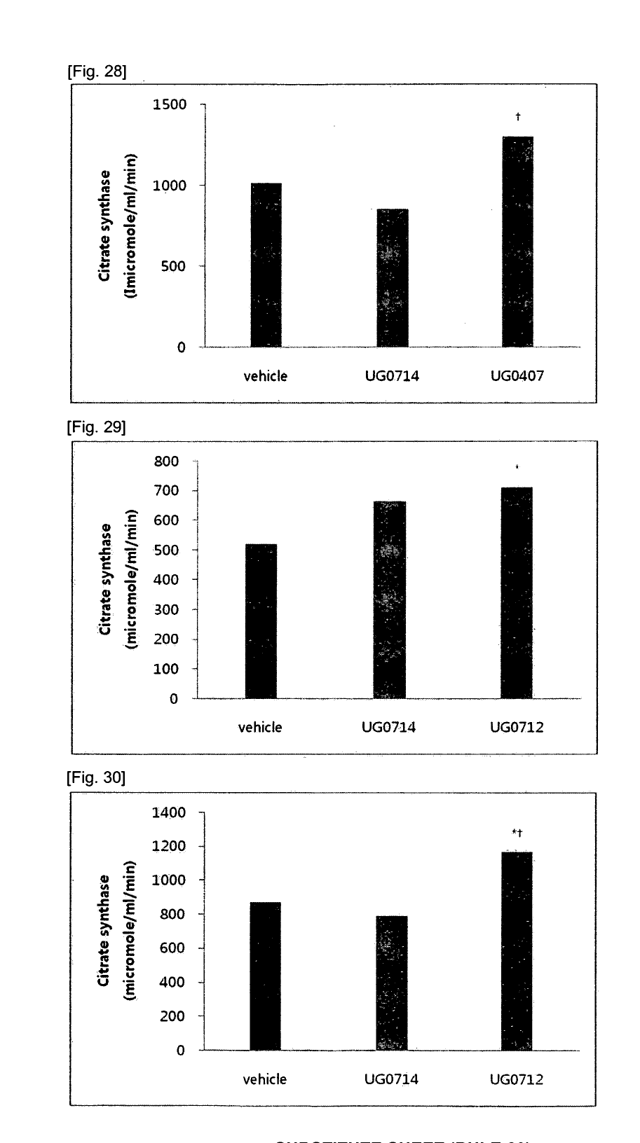

From the CS activity analyses, it is shown that the CS activity in the UG0407 treatment group increased in soleus (FIG. 28). It seems that UG0407 administration can increase CS activity relating to the energy generation via aerobic oxidation and thereby improving maximal oxygen consumption in the exercise group during exercising and helping the exercise performance, as shown in the results that the maximum running distance of the test group (UG0407 group) on the treadmill was longer, as compared with the exercise control group or positive control group.

2) UG0712

From the CS activity analyses, it is shown that the CS activity in the UG0712 treatment group increased in both non-exercise group and exercise group (FIGS. 29 and 30). It seems that UG0712 administration can increase CS activity relating to the energy generation via aerobic oxidation and thereby improving maximal oxygen consumption in the exercise group during exercising and helping the exercise performance, as shown in the results that the maximum running distance of the test group (UG0712 group) on the treadmill was longer, as compared with the exercise control group or positive control group.

### Example 4

### Measurement of Anti-oxidation effect

Oxygen free radical and reactive oxygen species (ROS) are generated during intensive physical exercise as well as in metabolic processes, and reported modify protein and DNA, and impair biomembranes, which results in significant damage to the cell structures or tissues in the body. Moreover, they are reported to cause cancers and adult diseases. Mitochondrion, peroxisome, and enzymes such as xanthine oxidase, NADPH oxidase, Cox (cyclooxygenase) existing in cell produce various ROS which causes oxidative damage. Reactive nitrogen species (RNS) are produced in a large amount by inflammatory response, and at the same time, ROS are also produced. The inflammatory response in muscle due to long-term or excessive exercises generates in- flammatory factor such as NO (nitric oxide).

Antioxidant system to remove such free radicals generated excessively can be classified into two categories: the first one includes an antioxidant enzymes such as SOD, glutathione peroxidase (GPx), and an endogenous non-enzymatic antioxidants such as antioxidant vitamins, glutathione, and the like, and the second one includes DNA repair enzymes for recovering the inner components of damaged DNAs.

To investigate anti-oxidation effect, NO analysis was performed in blood and muscle, SOD analysis was performed in hind lag muscle, and glutathione peroxidase activity in muscle was measured.

SOD (superoxide dismutase) inhibition rate was measured by using a commercially available SOD kit (superoxide dismutase Assays Designs, Catalog No. 30-023).

GPx (glutathione peroxidase) activity in muscle was analyzed by using Glutathione Peroxidase Activity kit (Assays Designs Cat. No. 900-158) for analysis of GPx through measuring change (reduction) of NADPH. The glutathione peroxidase activity was calculated according to the following formula:

$$\begin{matrix}Glutathione Peroxidase Activity = \frac{Δ A 340 / min}{0.00379 μ M - 1} \times \frac{0.2 ml}{Yml} \\ = n mol \text{/} min \text{/} ml \\ = Units \text{/} ml\end{matrix} \qquad [ Chem . 1 ]$$

TABLE 24
NO in blood of exercise group, collected before 2nd week

NO in blood (micromol/ml)

| | mean | SD |
|---|---|---|
| vehicle | 144 | 19.46 |
| UG0714 | 126 | 36.59 |
| UG0407 | 89 | 5.03 |
| vehicle: UG0714 | 0.2498567 | |
| vehicle: UG0407 | 0.0163008 | |
| UG0714: UG0407 | 0.1115504 | |

TABLE 25
NO in muscle of exercise group

soleus NO (micromole/mL)

| | mean | SD |
|---|---|---|
| Negative control | 8.9 | 1.78 |
| Positive control(UG0714) | 7.6 | 1.35 |
| UG0507 | 6.2 | 0.51 |

TABLE 26
NO in blood of non-exercise group

NO in blood (micromol/ml)

| W. No | mean | SD |
|---|---|---|
| Negative control | 84 | 8.30 |
| UG0714 | 79 | 6.09 |
| UG0712 | 62 | 15.36 |
| Negative control: UG0714 | 0.1747075 | |
| Negative control: UG0712 | 0.0287955 | |
| UG0714: UG0712 | 0.057481 | |

TABLE 27
NO in muscle of non-exercise group

NO-soleus (micromol/ml)

| W. No | mean | SD |
|---|---|---|
| Negative control | 6.02 | 0.46 |
| UG0714 | 6.20 | 0.80 |
| UG0712 | 5.35 | 0.44 |
| Negative control: UG0714 | 0.3357111 | |
| Negative control: UG0712 | 0.0233361 | |
| UG0714: UG0712 | 0.0404892 | |

TABLE 28
NO in blood of exercise group (the
blood collected before 2nd week)

NO in blood (micromole/mL)

| | mean | SD |
|---|---|---|
| Negative control | 144.28 | 19.46 |
| UG0714 | 126.00 | 36.59 |
| UG0712 | 100.50 | 27.67 |

TABLE 29

NO in blood (micromol/ml)

| | mean | SD |
|---|---|---|
| Negative control | 90 | 17.34 |
| UG0714 | 77 | 8.22 |
| UG0712 | 60 | 4.95 |
| Negative control: UG0714 | 0.16979548 | |
| Negative control: UG0712 | 0.04575625 | |
| UG0714: UG0712 | 0.02677473 | |

TABLE 30
NO in muscle of exercise group

NO (micromol/ml)
soleus

| | mean | SD |
|---|---|---|
| Negative control | 9 | 1.78 |
| UG0714 | 8 | 1.35 |
| UG0712 | 7 | 0.71 |
| Negative control: UG0714 | 0.1081975 | |
| Negative control: UG0712 | 0.027077 | |
| UG0714: UG0712 | 0.1523569 | |

NO (Nitric oxide) is synthesized from arginine under catalytic action of NOS (nitric oxide synthase). It has been known that blood flow in skeletal muscle is suppressed by presence of NOS inhibitor, and increase of blood flow in skeletal muscle suggests increase of NO level. Thus, the amount of NO in blood and muscle can act as an indirect marker of various oxidative stress factors in muscle.

1) UG0407

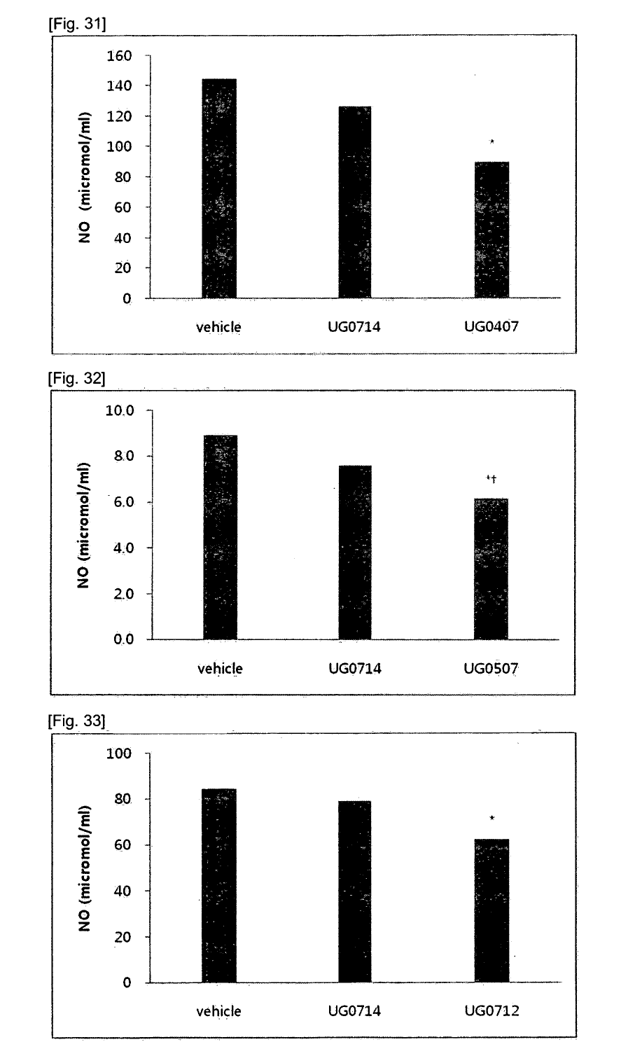

In the results obtained from the exercise group at 2nd week after administering the test materials, NO in blood of the UG0407 treatment group decreased statistically (FIG. 31). From the results, it can be known that the anti-stress factors were decreased by administering UG0407.

2) UG0507

In the NO analysis results obtained from the exercise group at 2nd week after administering UG0507, NO in muscle decreased statistically as compared with control group (FIG. 32). From the results, it can be known that the anti-stress factors were decreased by administering UG0507.

In the NO analysis results obtained from the exercise group at 8^{th} week after administering the test materials, NO concentrations in blood of non-exercise group was generally lower than those of exercise group. NO level of UG0712 treated non-exercise group was determined to 62±15.36 micromol/mL which was a statistically decreased value as compared with exercise control groups.

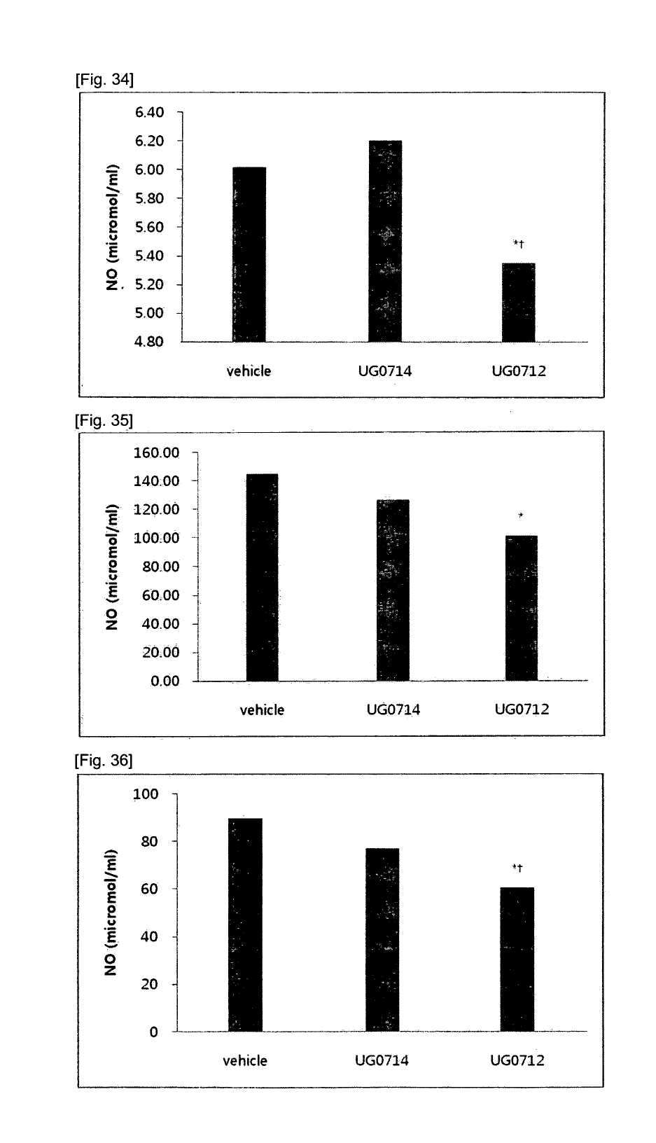

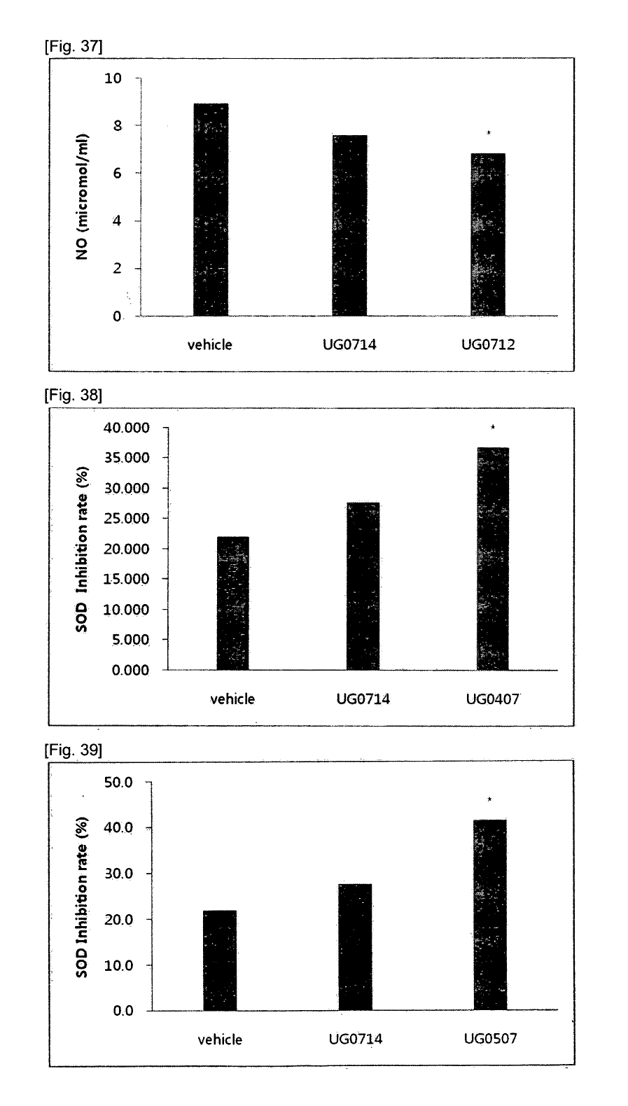

From the results in muscle, NO level of exercise group increased statistically as compared with those of non-exercise group, which was the same result as in blood NO analyses. The data obtained from UG0712 treated non-exercise group was 5±0.44 micromol/mL which was a statistically decreased value as compared with non-exercise control groups (p<0.05), and exercise control groups (p<0.01) (FIGS. 33 to 37). From the results, it can be known that the anti-stress factors were decreased by administering UG0712.

TABLE 31
SOD inhibition rate (%) in muscle of exercise group

SOD-Red inhibition (%)

| | mean | SD |
|---|---|---|
| vehicle | 21.960 | 7.24 |
| UG0714 | 27.568 | 9.79 |
| UG0407 | 36.701 | 9.56 |
| vehicle: UG0714 | 0.197598 | |
| vehicle: UG0407 | 0.0260894 | |
| UG0714: UG0407 | 0.1151487 | |

TABLE 32
SOD in muscle of exercise group

SOD inhibition rate(%)

| | mean | SD |
|---|---|---|
| Negative control | 22.0 | 7.24 |
| Positive control(UG0714) | 27.6 | 9.79 |
| UG0507 | 41.3 | 12.55 |

TABLE 33
SOD inhibition rate(%) in muscle of exercise group

SOD-Red inhibition(%)

| | mean | SD |
|---|---|---|
| Negative control | 22 | 7.24 |
| UG0714 | 28 | 9.79 |
| UG0712 | 31 | 6.10 |
| Negative control: UG0714 | 0.197598 | |
| Negative control: UG0712 | 0.0486405 | |
| UG0714: UG0712 | 0.2730523 | |

Superoxide dismutase (SOD) is one of the most important enzymes in anti-oxidative enzymatic system which can convert superoxide radical, the earliest product of aerobic exercise stage, into oxygen molecule and hydrogen peroxide. It has been used as a marker to the oxidative stress. SOD plays a role to prevent the generation of peroxynitrate, which is a powerful oxidative agent produced by reacting nitric oxide and superoxide (O²⁻). It was reported that SOD activity could be increased by regular exercising. Thus, anti-oxidation effect can be estimated by measuring SOD oxidation inhibition rate.

1) UG0407

In the results, SOD inhibition (%) of UG0407 treatment group increased statistically in muscle of the exercise group (FIG. 38). These results suggest that oxidation materials produced by oxidative stress can be effectively inhibited by administering UG0407.

2) UG0507

In the results, SOD inhibition (%) of UG0507 treatment group increased statistically in muscle of the exercise group (FIG. 39). These results suggest that oxidation materials produced by oxidative stress can be effectively inhibited by administering UG0507.

3) UG0712

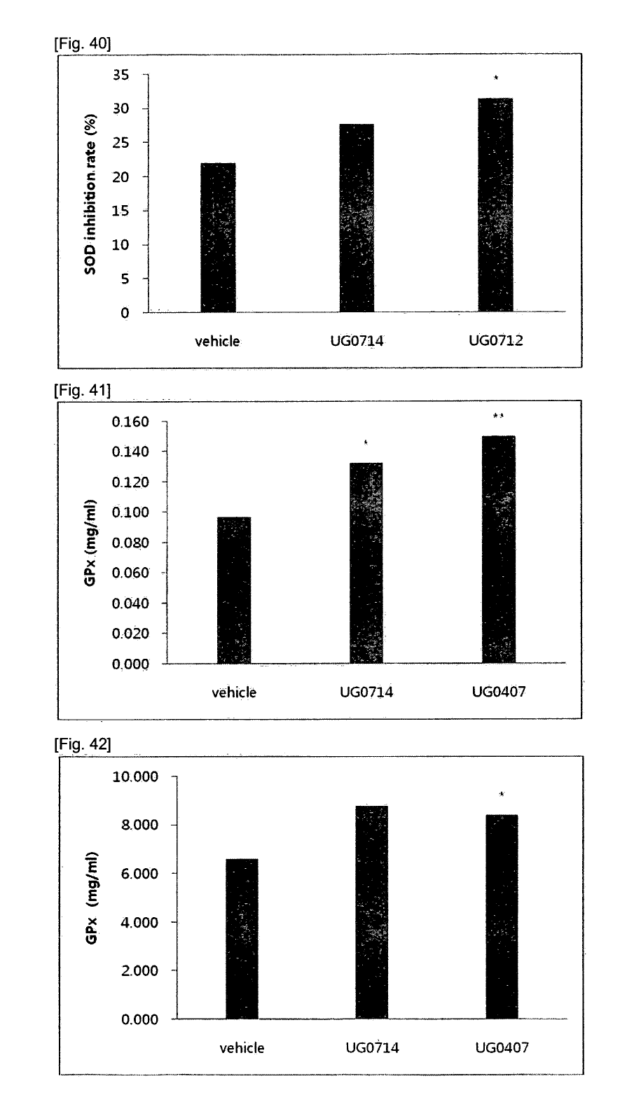

In the results, SOD inhibition (%) of UG0712 treatment group increased statistically in muscle of the exercise group (FIG. 40). These results suggest that oxidation materials produced by oxidative stress can be effectively inhibited by administering UG0712.

TABLE 34
GPx in muscle of non-exercise group

GPx-white
unit
protein(mg)

| | mean | SD |
|---|---|---|
| vehicle | 0.097 | 0.02 |
| UG0714 | 0.132 | 0.03 |
| UG0407 | 0.149 | 0.00 |
| vehicle: UG0714 | 0.0467973 | |
| vehicle: UG0407 | 0.001823 | |
| UG0714: UG0407 | 0.1629122 | |

TABLE 35
GPx in muscle of exercise group

GPx (mg/ml)

| | mean | SD |
|---|---|---|
| vehicle | 6.582 | 0.63 |
| UG0714 | 8.760 | 3.05 |
| UG0407 | 8.382 | 1.31 |
| vehicle: UG0714 | 0.0939291 | |
| vehicle: UG0407 | 0.0170736 | |
| UG0714: UG0407 | 0.4043888 | |

TABLE 36
GPx in liver of exercise group

GPx (mg) in liver protein

| | mean | SD |
|---|---|---|
| Negative control | 4.5 | 0.88 |
| Positive control(UG0714) | 5.6 | 2.20 |
| UG0507 | 12.3 | 1.80 |

TABLE 37
GPx in muscle of exercise group

GPx (mg/mL)

| | mean | SD |
|---|---|---|
| Negative control | 7 | 0.63 |
| UG0714 | 9 | 1.48 |
| UG0712 | 12 | 2.43 |
| Negative control: UG0714 | 0.04293813 | |
| Negative control: UG0712 | 0.005760453 | |
| UG0714: UG0712 | 0.023749648 | |

Glutathione peroxidase is one of anti-oxidation enzymes which have organ-protecting effect from oxidative injury and the anti-oxidative effect can be estimated by analyzing GPx activity in muscle.

1) UG0407

In the results, GPx in muscle of UG0407 treatment group increased statistically in both non-exercise and exercise groups, as compared with control groups (FIGS. 41 and 42). These results suggest that treatment of UG0407 can protect the organs effectively from oxidative injuries produced by exercise.

2) UG0507

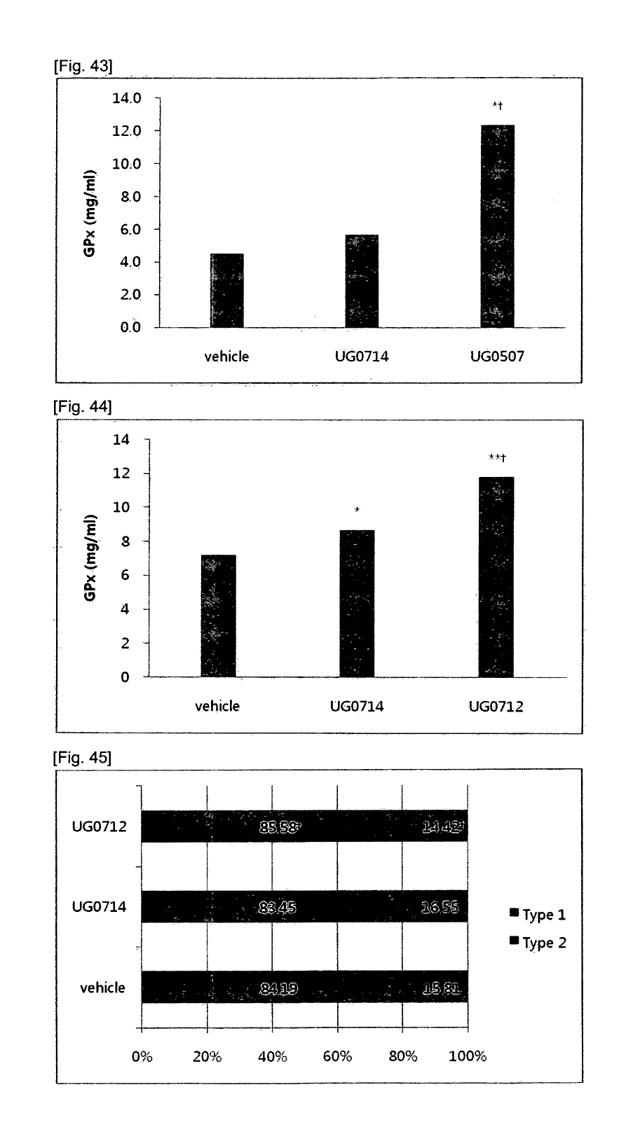

In the results, GPx in liver of UG0507 treatment group increased statistically in the non-exercise groups, as compared with control groups (FIG. 43). These results suggest that treatment of UG0507 can protect the organs effectively from oxidative injuries produced by exercise.

3) UG0712

In the results, GPx in muscle of UG0712 treatment group increased statistically in the exercise group, as compared with control group (FIG. 44). These results suggest that treatment of UG0712 can protect the organs effectively from oxidative injuries produced by exercise.

### Example 4

### ATPase Test

To investigate change of muscle fiber in hind leg's muscle relating to energy consumption, histochemical staining for myosin ATPase was performed and the results were used as auxiliary marker for exercise performance capacity.

1) Methods

The rats' muscle of left hind leg was frozen and cut to size of 12 μm by using microtome at 20° C. . The frozen-cut muscle samples were immediately stained with hemtoxylin-eosin, and serial section obtained from each block was fixed on the slide of microscope with checking the state of cellulose transfer. Myosin ATPase staining was performed by using acid preincubation. At least 200 fibers from each type of muscle from each animal were observed.

TABLE 38
ATPase test (%) (soleous)

| | ATPase Test (%) | | | |
|---|---|---|---|---|
| | Soleus | | Soleus | |
| | Type 1 | | Type 2 | |
| Negative control | mean | SD | mean | SD |
| Negative control | 84.19 | 2.35 | 15.81 | 2.35 |
| UG0714 | 83.45 | 0.63 | 16.55 | 0.63 |
| UG0712 | 85.58 | 1.55 | 14.42 | 1.55 |
| Negative control: UG0714 | 0.2238278 | | 0.22382782 | |
| Negative control: UG0712 | 0.1098226 | | 0.10982263 | |
| UG0714: UG0712 | 0.005025 | | 0.00502499 | |

TABLE 39
ATPase test (%) (Red gastrocnemius)

| | Red gastrocnemius | | Red gastrocnemius | |
|---|---|---|---|---|
| | Type 1 | | Type 2 | |
| | mean | SD | mean | SD |
| Negative control | 34.47 | 2.70 | 65.53 | 2.70 |
| UG0714 | 35.79 | 2.84 | 64.21 | 2.84 |
| UG0712 | 37.23 | 1.16 | 62.77 | 1.16 |
| Negative control: UG0714 | 0.1868666 | | 0.186866569 | |
| Negative control: UG0712 | 0.0184489 | | 0.018448946 | |
| UG0714: UG0712 | 0.10939 | | 0.109389958 | |

Muscle relating to exercise is divided by myosin ATPase staining into two subtypes, Type I fiber and Type II fiber.

Type I fiber aerobically uses glucose and fat as energy source and thus is strong to fatigue, and it is slow in contraction in aerobic energy metabolism, and so suitable to use long-term endurance exercise. Type I fiber is conventionally called as red muscle.

Type II fiber uses anaerobic non-oxygen energy metabolism and thus is weak to fatigue, and it is fast in contraction and so suitable to short-time and short-length exercise. Type II fiber is conventionally called as white muscle.

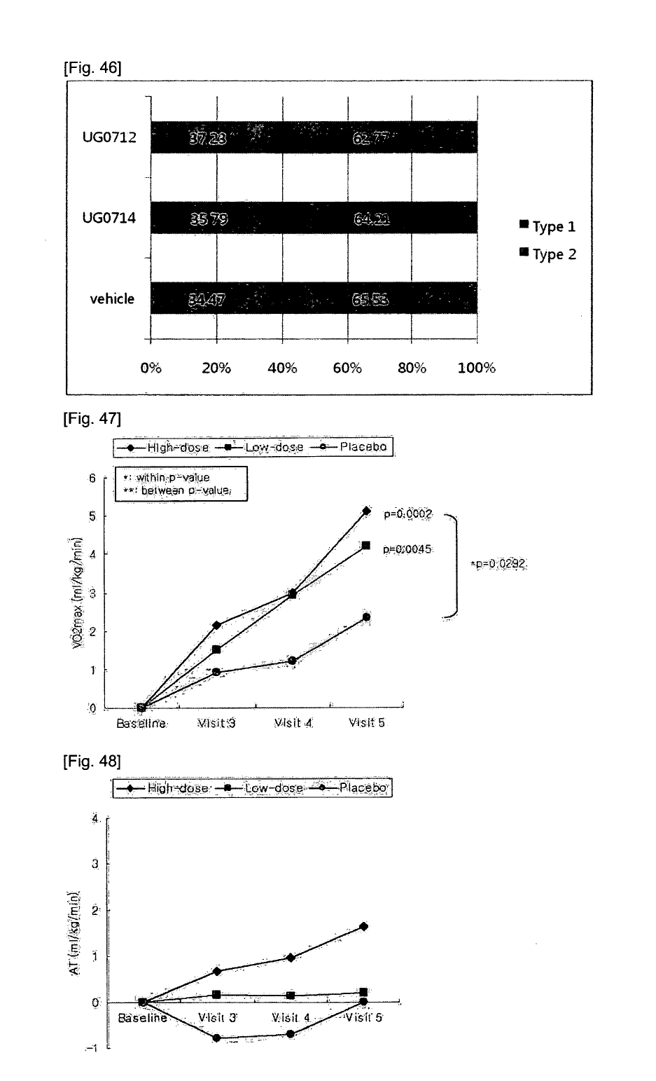

From the results of myosin ATPase histochemical staining to investigate change of type I fibers and type II fibers of the major hind leg muscles relating to exercise, the ratios of oxidative fibers type I in the exercise group were generally higher than those in the non-exercise group. In soleus, the ratios of type I fiber of UG0712 treated exercise group increased statistically as compared with that of non-exercise control group (p<0.01). The ratio of type I fibers of red gastrocnemius in the UG0712 treated exercise group increased statistically as compared with those in the exercise control group (p<0.05) (FIGS. 45 and 46).

Also, in the exercise group administered with test materials for 8 weeks with exercising, type I fibers increased slightly in general, as compared with those of non-exercise group. It is guessed that the muscular fibers proportion was changed to increase type I fibers in responding to the continuous exercise.

Accordingly, it is regarded that the tendency of higher ratio of oxidative fibers type I in the exercise group than those in the non-exercise group was from that the continuous exercise directed the metabolism of muscle to increase oxidative capacity and delay muscle fatigue state.

Such tendency further increased by the UG0712 administration, and the exercise capacity on the treadmill was guessed to increase for that reason.

### Example 5

### Evaluation of Exercise Capacity Improvement Effect in Human (VO₂ Max and AT Measurements) and Safety Test

(1) Methods

Single centered, double-blinded, randomly-allocated, and placebo controlled study was performed.

Healthy people over 20 years old who had not exercised regularly for 3 months before the date of the clinical trial, were designated to subjects. Total number of subjects was 123, and the number of subjects who completed the clinical trial was 82. The subjects were randomly allocated to UG0712 high dose group, UG0712 low dose group, and placebo group, respectively, and the study was performed in a double-blind manner.

For UG0712 high dose group, total 500 mg of UG0712 was administered per day (each dose of 250 mg, twice a day). For UG0712 low dose group, total 100 mg of UG0712 was administered per day (each dose of 50 mg, twice a day). For placebo group, total 500 mg of carboxymethylcellulose (CMC) was administered per day (each dose of 250 mg, twice a day).

The administration period was 12 weeks, and subjects performed a given exercise (three times a week, 60 to 90 min aerobic exercise and resistive exercise per each time of exercise). Aerobic exercise was performed by using treadmill and ergometer in a strength of 70 to 80% VO₂ max.

At the day of test materials' administration, and at 4^{th} week, 8^{th} week and 12th week after the start day of administration, VO₂ max and AT were estimated, and safety test was performed.

(2) Measurement of VO₂ max

To estimate the effect of exercise capacity improvement, VO₂ max was measured.

From the VO₂ max (the amount of maximal oxygen consumption) analyses results for all the subjects, the mean value of change (Change 3) to baseline in the last visit (Visit 5) of high dose group was 5.11±4.81 ml/kg/min, that of low dose group was 4.20±5.49 ml/kg/min, and that of placebo group was 2.34±2.99 ml/kg/min. Two UG0712 treatment groups showed statistically increased value according to visit number, as compared with placebo group (RM ANOVA, p=0.0002 in high dose group, p=0.0045 in low dose group). The differences of Visit 3, 4 and 5 from baseline in two UG0712 treatment groups were generally higher than those of placebo group, and in particular, the values of high dose group were statistically different from those of placebo group (RM ANCOVA, p=0.0292) (Table 40, FIG. 47)

TABLE 40
Measurement of Exercise performance (VO₂ max) (Unit = ml/kg/min)(ITT)

| Treatment | Visit | N | Mean | SD | Median | Min | Max | P-value¹⁾ |
|---|---|---|---|---|---|---|---|---|
| High-dose | Baseline | 39 | 28.64 | 4.87 | 27.73 | 20.61 | 39.72 | 0.0002 |
| | Visit 3 | 39 | 30.78 | 5.22 | 30.96 | 20.58 | 42.50 | |
| | Visit 4 | 39 | 31.62 | 4.95 | 31.18 | 20.80 | 41.13 | |
| | Visit 5 | 39 | 33.74 | 4.88 | 34.20 | 20.80 | 44.53 | |
| | Change 1 | 39 | 2.15 | 3.51 | 1.86 | −4.27 | 9.88 | |
| | Change 2 | 39 | 2.98 | 4.17 | 3.02 | −7.51 | 11.21 | |
| | Change 3 | 39 | 5.11 | 4.81 | 5.15 | −5.94 | 19.63 | |
| Low-dose | Baseline | 39 | 29.09 | 4.74 | 28.72 | 20.38 | 40.65 | 0.0045 |
| | Visit 3 | 39 | 30.61 | 5.12 | 30.63 | 20.38 | 40.65 | |
| | Visit 4 | 39 | 32.03 | 5.28 | 31.81 | 20.38 | 40.65 | |
| | Visit 5 | 39 | 33.28 | 6.02 | 33.00 | 19.05 | 45.39 | |
| | Change 1 | 39 | 1.52 | 2.72 | 0.00 | −3.08 | 8.52 | |
| | Change 2 | 39 | 2.94 | 4.23 | 1.27 | −3.08 | 15.16 | |
| | Change 3 | 39 | 4.20 | 5.49 | 2.84 | −6.60 | 18.51 | |
| Placebo | Baseline | 39 | 30.42 | 6.73 | 29.71 | 20.33 | 49.89 | 0.4735 |
| | Visit 3 | 39 | 31.34 | 6.32 | 30.34 | 21.40 | 51.50 | |
| | Visit 4 | 39 | 31.63 | 6.62 | 30.33 | 20.00 | 51.50 | |
| | Visit 5 | 39 | 32.77 | 6.63 | 31.27 | 21.40 | 51.50 | |
| | Change 1 | 39 | 0.92 | 3.65 | 0.14 | −8.04 | 10.08 | |
| | Change 2 | 39 | 1.21 | 3.12 | 1.19 | −5.12 | 7.70 | |
| | Change 3 | 39 | 2.34 | 2.99 | 1.61 | −5.12 | 8.63 | |

| | | |
|---|---|---|
| P-value²⁾ | High-dose vs Placebo | 0.0292 |
| | Low-dose vs Placebo | 0.2537 |

¹⁾Change over time: RM ANOVA
²⁾Difference between treatment groups: RM ANCOVA (Dunnett's multiple comparison)
Change 1: Visit 3 − Baseline, Change 2: Visit 4 − Baseline, Change 3: Visit 5 − Baseline

Aerobic capacity of individual is defined as the maximum volume of oxygen that can be consumed by individual's muscle during maximal or exhaustive exercise. To measure maximal aerobic capacity, VO₂ max test can be performed. VO₂ max can be recognized as the functional capacity of each individual and is an important factor for the lung's oxygen delivery capacity to blood vessel, cardiac blood pumping action and procedure for supplying pumped blood to muscle.

From the results, VO₂ max which represents an aerobic exercise capacity according to the amount of maximal oxygen consumption, i.e., endurance capacity of cardiopulmonary exercise endurance, increased statistically in the high dose of UG0712 treatment group as compared with the placebo group (RM ANOCOVA, VO₂ max p=0.0292).

(3) AT (Anaerobic Threshold)

To estimate the effect of exercise capacity improvement, anaerobic threshold (AT) was measured.

From the AT analyses results for all tested subjects (ITT groups), the mean value of change (Change 3) to baseline in the last visit of high dose group was 1.63±4.18 ml/kg/min, that of low dose group was 0.19±3.59 ml/kg/min, and that of placebo group was -0.01±4.74 ml/kg/min. Two UG0712 treatment groups showed statistically increased value according to visit number, as compared with placebo group. The differences of Visit 3, 4 and 5 from baseline in two UG0712 treatment groups were generally higher than those of placebo group, and in particular, the values of high dose group were statistically different from those of placebo group (RM ANCOVA, p=0.0378) (Table 41, FIG. 48)

TABLE 41
Measurement of Exercise performance (AT) (Unit = ml/kg/min) (ITT)

| Treatment | Visit | N | Mean | SD | Median | Min | Max | P-value¹⁾ |
|---|---|---|---|---|---|---|---|---|
| High-dose | Baseline | 39 | 19.28 | 4.23 | 18.75 | 9.55 | 32.00 | 0.2476 |
| | Visit 3 | 39 | 19.94 | 3.63 | 19.48 | 13.26 | 28.24 | |
| | Visit 4 | 39 | 20.23 | 2.85 | 20.74 | 15.73 | 28.59 | |
| | Visit 5 | 39 | 20.91 | 3.47 | 20.40 | 15.76 | 28.65 | |
| | Change 1 | 39 | 0.66 | 4.03 | 0.54 | −9.71 | 6.96 | |
| | Change 2 | 39 | 0.95 | 3.93 | 1.21 | −11.84 | 8.31 | |
| | Change 3 | 39 | 1.63 | 4.18 | 0.93 | −9.75 | 9.93 | |
| Low-dose | Baseline | 39 | 18.83 | 3.46 | 19.03 | 12.87 | 29.04 | 0.9956 |
| | Visit 3 | 39 | 18.96 | 3.13 | 19.18 | 12.94 | 29.04 | |
| | Visit 4 | 39 | 18.96 | 3.46 | 19.18 | 12.94 | 29.04 | |
| | Visit 5 | 39 | 19.02 | 3.72 | 19.18 | 10.43 | 29.04 | |
| | Change 1 | 39 | 0.14 | 1.96 | 0.00 | −4.36 | 4.88 | |
| | Change 2 | 39 | 0.13 | 2.89 | 0.00 | −5.64 | 6.76 | |
| | Change 3 | 39 | 0.19 | 3.59 | 0.00 | −7.45 | 7.90 | |
| Placebo | Baseline | 39 | 20.03 | 5.11 | 19.41 | 12.95 | 32.91 | 0.7681 |
| | Visit 3 | 39 | 19.23 | 3.98 | 18.68 | 12.57 | 29.52 | |
| | Visit 4 | 39 | 19.32 | 3.56 | 18.86 | 12.58 | 29.07 | |
| | Visit 5 | 39 | 20.02 | 4.86 | 19.08 | 10.66 | 36.09 | |
| | Change 1 | 39 | −0.80 | 4.69 | 0.00 | −13.11 | 12.44 | |
| | Change 2 | 39 | −0.71 | 4.62 | 0.00 | −13.46 | 7.83 | |
| | Change 3 | 39 | −0.01 | 4.74 | 0.00 | −13.46 | 9.60 | |

| | | |
|---|---|---|
| P-value²⁾ | High-dose vs Placebo | 0.0378 |
| | Low-dose vs Placebo | 0.9626 |

¹⁾Change over time: RM ANOVA
²⁾Difference between treatment groups: RM ANCOVA (Dunnett's multiple comparison)
Change 1: Visit 3 − Baseline, Change 2: Visit 4 − Baseline, Change 3: Visit 5 − Baseline

The anaerobic threshold is the specific point at which lactic acid concentration in blood starts to increase according to the increase of exercise intensity. If AT level is high, anaerobic metabolism does not occur and aerobic exercise can be performed for a long time. It means that individual can exercise continuously for a long time, keeping his/her own exercise capacity pace.

From the results, AT representing aerobic exercise capacity according to anaerobic threshold increased statistically in the UG0712 high dose treatment group as compared with placebo group (AT p=0.0378).

VO₂ max and AT are independent markers of aerobic exercise capacity, i.e., improvement of cardiopulmonary endurance capacity. From the above results, VO₂ max and AT values in the UG0712 high dose treatment group statistically increased as compared with placebo group, and thus it can be confirmed that exercise capacity and endurance capacity of normal adult can be improved through the improvement of aerobic exercise capacity by administration of high dose UG712 (500mg/day).

(4) Safety Test

1) Methods

The results of all the randomly allocated 117 subjects were used for safety test since

UG0712 or placebo was administered to all the subjects and at least one safety data for all the subjects were presented and could be analyzed.

(a) Abnormal response

Abnormal response through conscious/unconscious symptom was estimated from the date of administration of test materials to 12^{th} week (visit 5). If any abnormal response occurred, its symptom, occurrence time, intensity and cause and effect were recorded. The abnormal response was recorded by subjects' spontaneous report or by medical interview check at the time of visit. The abnormal clinical experimental test and vital sign results which are clinically remarkable, were also recorded.

(b) General Manifestations

Vital sign, i.e., blood pressure (mmHg) and pulse (#/min) were measured after stabilizing the subjects at least for 5 min. The laboratory test and physical examination manifestation were conducted at screening visit (visit 1), visits 3, 4 and 5, and the results were recorded. Among the above factors, if clinically remarkable abnormal symptoms were occurred, such results were recorded in detail.

2) Results

In the laboratory test, vital sign and physical examination, there were no remarkable changes before and after the clinical trial. Comparing the occurrence rate of abnormal symptoms, those of treatment groups and those of placebo group were not different sta- tistically. Accordingly, it can be known that UG0712 preparation can be safely used.

1-22. (canceled)

23. A method for improving exercise performance and fatigue recovery comprising administering to a subject in need thereof a composition comprised of an extract of the leaves from a plant of the genus Panax or a processed product of said extract or a mixture thereof.

24. (canceled)

25. A method for enhancing VO2 max, AT (anaerobic threshold) or citrate synthase activity said method comprising administering to a subject in need thereof a composition comprised of an extract of the leaves from a plant of the genus Panax or a processed product of said extract or a mixture thereof.

26-27. (canceled)

28. The method according to claim 23, wherein said composition comprises 3-O-glycosides of protopanaxatriol and 3-O-glycosides of protopanaxadiol.

29. The method according to claim 28, wherein the ratio of 3-O-glycosides of protopanaxatriol : 3-O-glycosides of protopanaxadiol in said leaf extract is 1:0.1 to 1.

30. The method according to claim 28, wherein the ratio of 3-O-glycosides of protopanaxatriol : 3-O-glycosides of protopanaxadiol in said processed leaf extract or mixture of said leaf extract and said processed leaf extract is 1:0.5 to 1.5.

31. (canceled)

32. The method according to claim 23, wherein said composition contains one or more ginsenosides in an amount of 30 wt % or more in total.

33. (canceled)

34. The method according to claim 23, wherein said composition comprises one or more ginsenoside(s) selected from the group consisting of Rg3, Rg5 and Rk1.

35. The method according to claim 34, wherein said leaf extract contains more than 1.5 wt % of Rg3, Rg5 and Rk1 in total.

36. The method according t claim 34, wherein said processed leaf extract, or mixture of said leaf extract and said leaf extract contains more than 10 wt % of Rg3, Rg5 and Rk1 in total.

37. The method according claim 23, wherein said plant of the genus Panax is selected from the group of species consisting of Panax ginseng, Panax japonicum, Panax quinquefolium, Panax notoginseng, Panax trifolium, Panax pseudoginseng Panax vietnamensis, Panax elegatior, Panax wangianus and Panax bipinratifidus.

38. The method according to claim 23, wherein the mixing ratio of said plant leaf extract: processed plant leaf extract in the mixture is 1:0.1 to 5.

39. (canceled)

40. The method according to claim 23, further comprising one or more components selected from the group consisting of squalene, Saururus chinensis aqueous extract, Acanthopanax sessiliflorus aqueous extract, aqueous extract of Cordycepsmilitaris and Paecilomyces japonica, cola nut powder or extract, vitamins, minerals, taurine, creatine, phosphatidylcholine, glutamine, L-arginine and L-carnitine.

41-47. (canceled)

48. The method according to claim 25, wherein said composition comprises 3-O-glycosides of protopanaxatriol and 3-O-glycosides of protopanaxadiol.

49. The method according to claim 48, wherein the ratio of 3-O-glycosides of protopanaxatriol: 3-O-glycosides of protopanaxadiol in said leaf extract is 1:0.1 to 1.

50. The method according to claim 48, wherein the ratio of 3-O-glycosides of protopanaxatriol: 3-O-glycosides of protopanaxadiol in said processed leaf extract or mixture of said leaf extract and said processed leaf extract is 1:0.5 to 1.5.

51. The method according to claim 25, wherein said composition contains one or more ginsenosides in an amount of 30 wt % or more in total.

52. The method according to claim 25, wherein said composition comprises one or more ginsenoside(s) selected from the group consisting of Rg3, Rg5 and Rk1.

53. The method according to claim 52, wherein said leaf extract contains more than 1.5 wt % of Rg3, Rg5 and Rk1 in total.

54. The method according to claim 52, wherein said processed leaf extract, or mixture of said leaf extract and said processed leaf extract contains more than 10 wt % of Rg3, Rg5 and Rk1 in total.

55. The method according to claim 25, wherein said plant of the genus Panax is selected from the group of species consisting of Panax ginseng, Panax japonicum, Panax quinquefolium, Panax notoginseng, Panax trifolium, Panax pseudoginseng Panax vietnamensis, Panax elegatior, Panax wangianus and Panax bipinratifidus.

56. The method according to claim 25, wherein the mixing ratio of said plant leaf extract: processed plant leaf extract in the mixture is 1:0.1 to 5.

57. The method according to claim 25, further comprising one or more components selected from the group consisting of squalene, Saururus chinensis aqueous extract, Acanthopanax sessiliflorus aqueous extract, aqueous extract of Cordycepsmilitaris and Paecilomyces japonica, cola nut powder or extract, vitamins, minerals, taurine, creatine, phosphatidylcholine, glutamine, L-arginine and L-carnitine.
